(19) United States
(12) Patent Application Publication
(10) Pub. No.: US 2009/0169425 A1
(43) Pub. Date: Jul. 2, 2009

(54) APPARATUS AND METHOD FOR DISINFECTING FOOD USING PHOTO-CATALYTIC REACTION OF TITANIUM DIOXIDE AND ULTRAVIOLET RAYS

(75) Inventors: Ji Yong PARK, Goyang (KR); Yong Joo KIM, Goyang (KR); Young Bong KIM, Seoul (KR); Yun Sik CHOI, Incheon (KR); Mi Hee CHO, Ansan (KR); Yong Ki SEO, Seoul (KR); Jin Hee LEE, Seoul (KR)

Correspondence Address: EDWARDS ANGELL PALMER & DODGE LLP, P.O. BOX 55874, BOSTON, MA, 02205, US

(73) Assignees: INDUSTRY-ACADEMIC COOPERATION FOUNDATION, YONSEI UNIVERSITY, Seoul (KR); CJ CheilJedang Corporation, Seoul (KR)

(21) Appl. No.: 11/965,338

(22) Filed: Dec. 27, 2007

(51) Int. Cl. A61L 2/232; A61L 2/10

(52) U.S. Cl. 422/29; 422/186.3

(57) ABSTRACT

The present invention discloses an apparatus and method for disinfecting an object in a batch, continuous, or mixed mode. The apparatus according to the present invention comprises a bath to/from which water is flowed in/drained out; and at least one ultraviolet ray (UV) lamp unit assembly mounted in the bath, each UV lamp unit assembly including a plurality of UV lamp units. Each UV lamp unit comprises a quartz tube, a UV lamp mounted in the quartz tube and a photo-catalyst layer of titanium dioxide formed on an outer surface of the quartz tube to disinfect an object in the bath by the photo-catalytic reaction of titanium dioxide and UV. The apparatus of the present invention may comprise a conveyer device comprising a driving roller, driven rollers and a conveyer belt wound around the rollers, the conveyer device is divided into an inlet portion formed at a first outside of the bath, a conveying portion formed in water in the bath and a discharging portion formed at a second outside of the bath.

### TECHNICAL FIELD

The present invention relates to an apparatus and a method for disinfecting foods using a photo-catalyst of titanium dioxide, in which foods can be disinfected by titanium dioxide photo-catalytic reaction under ultraviolet irradiation.

### BACKGROUND ART

Recently, the demand for safe foods is rapidly increasing as the diet has been greatly changed to the consumption of natural, organic and raw-eating foods. However, such natural or organic foods can be easily contaminated with microorganisms, and thus safety issues such as outbreaks of food-borne illness have focused attention on effective disinfecting methods.

Photo-catalytic disinfection that uses titanium dioxide has been widely used for purification of water and air since its antimicrobial effect has been proved. However there was almost no trial applying the titanium dioxide photo-catalyst disinfection technique to foods. This technique has advantages such as moderate costs, low energy consumption and semi-permanence. In addition, titanium dioxide is used as a food additive and is non-toxic, chemically stable, and capable of repeated use without loss of catalytic activity. Therefore, the titanium dioxide photo-catalyst reaction is applicable for a non-thermal disinfection method for the food industry.

The titanium dioxide photo-catalyst reaction generates strong oxidizing power when illuminated with ultraviolet (UV) light. Electron-hole pairs, an electron in a conduction band (e⁻_{cb}) and a hole in a valence band (h⁺_{vb}), are generated on a surface of the titanium dioxide photo-catalyst by UV illumination and photon absorption. The e⁻_{cb} generates hydroxyl radicals through a reductive pathway. The e⁻_{cb} reduces oxygen to a superoxide radical, followed by a subsequent reduction to hydrogen peroxide and finally to a hydroxyl radical. The h⁺_{vb} generates hydroxyl radicals through an oxidative pathway. The h⁺_{vb} reacts with hydroxyl ions or water to form the hydroxyl radical, which decomposes organic compounds and causes damage to microorganisms.

In conventionally used methods, a large quantity of titanium dioxide powder is added to a solution. These methods have an advantage of providing a large reaction area which can be used in the photo-catalyst reactions. But, the bactericidal effect is reduced since UV transmittance is interrupted by titanium dioxide powder itself, and there is a limitation to practical applications due to difficulty of subsidiary processes such as recovery, separation and recycling of catalysts.

Various methods to immobilize the titanium dioxide were investigated to solve the limitation. However, conventional techniques have many problems in a method of immobilizing titanium dioxide, so that UV transmittance is remarkably lowered and bactericidal efficiency is reduced.

Japanese Patent Application Publication No. 2002-219456 discloses an apparatus for disinfecting agricultural water, waste water, and the like, by inserting several sheets of lattice nets coated with titanium dioxide around UV lamps. However, there is a problem that several separate nets should be equipped. In addition, there are a lot of drawbacks that the bactericidal efficiency is reduced over time, the extra works such as exchanging the lamp or cleaning the apparatus are required, and bactericidal capacity is reduced as the distance between the lattice net and UV is increases.

The above information disclosed in this Background section is only for enhancement of understanding of the background of the invention and therefore it may contain information that does not form the prior art that is already known in this country to a person of ordinary skill in the art.

### SUMMARY OF THE DISCLOSURE

The present invention is intended for solving the problems as above. An object of the present invention is to provide an apparatus and a method for disinfecting foods, which is able to improve function of disinfecting foods, using ultraviolet generated from an ultraviolet lamp and a photo-catalyst of titanium dioxide.

Another object of the present invention is to provide an apparatus and a method for disinfecting foods in a batch, continuous or mixed mode.

In order to achieve the above objects, the apparatus for disinfecting an object (for example, fruits and vegetables) according to the present invention comprises a bath to/from which water is flowed/drained out; and at least one ultraviolet (UV) lamp unit assembly mounted in the bath. The UV lamp unit assembly each includes a plurality of UV lamp units. Here, each UV lamp unit comprises a quartz tube, a UV lamp mounted in the quartz tube and a photo-catalyst layer of titanium dioxide formed on an outer surface of the quartz tube to disinfect the object in the bath by the photo-catalytic reaction of titanium dioxide and UV.

In the above apparatus, the bath can comprise a case in which water is accommodated and a cover mounted openably/closably to the case, and the UV lamp unit assembly is provided at a lower portion of the bath. At this time, it is preferable that an additional UV lamp unit assembly provided on an inner surface of the cover.

In addition, the apparatus according to the present invention further comprises an air-blowing means including an air flow line provided below the UV lamp unit assembly, an air supplying unit installed outside the bath and a connecting line connecting the air flow line and the air supplying unit so as to blow air into water in the bath through openings formed on the air flow line.

The apparatus of the present invention may comprise: a base having a plurality of guide channels fixed thereon and a driving means for reciprocating the bath linearly; a plurality of roller fixed rotatably on a lower surface of the bath and received in the guide channels of the base; and a fixture connected to the driving means of the base.

In addition, the apparatus of the present invention may comprises a conveyer device comprising a driving roller, driven rollers and a conveyer belt wound around the rollers, the conveyer device being divided into an inlet portion formed at a first outside of the bath, a conveying portion formed in water in the bath and a discharging portion formed at a second outside of the bath, wherein the bath has openings formed on the first and second side walls thereof for enabling the conveyer and object putted on the conveyer to be passed through the openings, and the UV lamp unit assembly is disposed below the conveyer belt in the bath.

In this structure, it is preferable that the UV lamp unit assembly is disposed between an upper portion and a lower portion of the conveyer belt and the conveyer belt includes a pair of supporting bodies spaced apart from each other and a mesh sheet secured to the supporting bodies, and the supporting bodies are supported on idle roller provided rotatably at walls of the bath.

In particular, the apparatus of the present invention can further comprises a first conveyer device provided in the bath and a second conveyer device placed below the first conveyer device, each of the first and second conveyer devices comprising a driving roller, driven rollers and a conveyer belt wound around the rollers, and each conveyer device being divided into an inlet portion formed at a first outside of the bath, a conveying portion formed in water in the bath and a discharging portion formed at a second outside of the bath.

Here, the bath has openings formed on the first and second side walls thereof for enabling the conveyer and object putted on the conveyer to be passed through the openings, and each UV lamp unit assembly is disposed below the corresponding conveyer belt in the bath.

At this time, each of the UV lamp unit assemblies is disposed between an upper portion and a lower portion of the corresponding conveyer belt, and the conveyer belt of each conveyer device includes a pair of supporting bodies spaced apart from each other and a mesh sheet secured to the supporting bodies, and the supporting bodies are supported on idle roller provided at walls of the bath.

The above features and advantages of the present invention will be apparent from or are set forth in more detail in the accompanying drawings, which are incorporated in and form a part of this specification, and the following Detailed Description, which together serve to explain by way of example the principles of the present invention.

### BRIEF DESCRIPTION OF THE DRAWINGS

FIG. 1 is a sectional view of an ultraviolet lamp unit employed in an apparatus for disinfecting food according to the present invention;

FIG. 2a and FIG. 2b are scanning electron microscopy photographs of a quartz tube on which a titanium dioxide photo-catalyst layer is not formed and a quartz tube having a photo-catalyst layer of titanium dioxide formed thereon;

FIG. 3A is a prospective view of an apparatus for disinfecting food according to the first embodiment of the present invention;

FIG. 3B is a front sectional view of an apparatus for disinfecting food shown in FIG. 3B;

FIG. 4 is a view illustrating an apparatus for disinfecting food according to the second embodiment of the present invention;

FIG. 5 is a view illustrating an apparatus for disinfecting food according to the third embodiment of the present invention;

FIG. 6 is a view illustrating an apparatus for disinfecting food according to the fourth embodiment of the present invention;

FIG. 7 is a plane view of a conveyer belt constituting a conveyer device shown in FIG. 5 and FIG. 6;

FIG. 8 is a graph representing the bactericidal effect against Escherichia coli over time, in the disinfecting apparatus, depending on whether or not a photo-catalyst layer of titanium dioxide is formed on the quartz tube (▴: when ultraviolet is irradiated in E. coli, using a quartz tube, wherein no photo-catalyst layer of titanium dioxide is formed on its surface, as a protective tube; ▪: when ultraviolet is irradiated in E. coli, using a quartz tube, wherein a photo-catalyst layer of titanium dioxide is formed on its surface, as a protective tube; and I (error bar): indicating latent error amount related to each datum signal of data series);

FIG. 9 is a graph representing the bactericidal effect against Listeria monocytogenes over time, in the disinfecting apparatus, depending on whether or not a photo-catalyst layer of titanium dioxide is formed on the quartz tube (▴: when ultraviolet is irradiated in Listeria monocytogenes, using a quartz tube, wherein no photo-catalyst layer of titanium dioxide is formed on its surface, as a protective tube; ▪: when ultraviolet is irradiated in Listeria monocytogenes, using a quartz tube, wherein a photo-catalyst layer of titanium dioxide is formed on its surface, as a protective tube; and I (error bar): indicating latent error amount related to each datum signal of data series);

FIG. 10 is a graph representing the bactericidal effect against Bacillus cereus over time, in the disinfecting apparatus, depending on whether or not a photo-catalyst layer of titanium dioxide is formed on the quartz tube (▴: when ultraviolet is irradiated in Bacillus cereus, using a quartz tube, wherein no photo-catalyst layer of titanium dioxide is formed on its surface, as a protective tube; ▪: when ultraviolet is irradiated in Bacillus cereus, using a quartz tube, wherein a photo-catalyst layer of titanium dioxide is formed on its surface, as a protective tube; and I (error bar): indicating latent error amount related to each datum signal of data series);

FIG. 11 is a graph representing the bactericidal effect against Salmonella typhimurium over time, in the disinfecting apparatus, depending on whether or not a photo-catalyst layer of titanium dioxide is formed on the quartz tube (▴: when ultraviolet is irradiated in Salmonella typhimurium, using a quartz tube, wherein no photo-catalyst layer of titanium dioxide is formed on its surface, as a protective tube; ▪: when ultraviolet is irradiated in Salmonella typhimurium, using a quartz tube, wherein a photo-catalyst layer of titanium dioxide is formed on its surface, as a protective tube; and I (error bar): indicating latent error amount related to each datum signal of data series);

FIG. 12 is a graph representing the bactericidal effect against Bacillus subtilis spore over time, in the disinfecting apparatus, depending on whether or not a photo-catalyst layer of titanium dioxide is formed on the quartz tube (▴: when ultraviolet is irradiated in Bacillus subtilis spores, using a quartz tube, wherein no photo-catalyst layer of titanium dioxide is formed on its surface, as a protective tube; ▪: when ultraviolet is irradiated in Bacillus subtilis spores, using a quartz tube, wherein a photo-catalyst layer of titanium dioxide is formed on its surface, as a protective tube; and I (error bar): indicating latent error amount related to each datum signal of data series);

FIG. 13 is a photograph of viewing change in the outer shape of Escherichia coli with the photo-catalytic reaction via scanning electron microscope;

FIG. 14 is a photograph of viewing change in the outer shape of Listeria moncytogenes with the photo-catalytic reaction via scanning electron microscope;

FIG. 15 is a photograph of viewing change in the outer shape of Bacillus cereus with the photo-catalytic reaction via scanning electron microscope;

FIG. 16 is a photograph of viewing change in the outer shape of Salmonella typhimurium with the photo-catalytic reaction via scanning electron microscope;

FIG. 17 is a photograph of viewing change in the outer shape of Bacillus subtilis spore with the photo-catalytic reaction via scanning electron microscope;

FIG. 18 is a graph representing the bactericidal effect against total aerobic bacteria in carrot over time, in the disinfecting apparatus, depending on whether or not a photo-catalyst layer of titanium dioxide is formed on the quartz tube (♦: when carrot is immersed in general tap water; [symbol] when ultraviolet is irradiated in carrot, using a quartz tube, wherein no photo-catalyst layer of titanium dioxide is formed on its surface, as a protective tube; ▴: when ultraviolet is irradiated in carrot, using a quartz tube, wherein a photo-catalyst layer of titanium dioxide is formed on its surface, as a protective tube; and I (error bar): indicating latent error amount related to each datum signal of data series);

FIG. 19 is a graph representing the bactericidal effect against total aerobic bacteria in Angelica keiskei over time, in the disinfecting apparatus, depending on whether or not a photo-catalyst layer of titanium dioxide is formed on the quartz tube (▴: when ultraviolet is irradiated in Angelica keiskei, using a quartz tube, wherein no photo-catalyst layer of titanium dioxide is formed on its surface, as a protective tube; ▪: when ultraviolet is irradiated in Angelica keiskei, using a quartz tube, wherein a photo-catalyst layer of titanium dioxide is formed on its surface, as a protective tube; and I (error bar): indicating latent error amount related to each datum signal of data series);

FIG. 20 is a graph representing the bactericidal effect against total aerobic bacteria in iceberg lettuce over time, in the disinfecting apparatus, depending on whether or not a photo-catalyst layer of titanium dioxide is formed on the quartz tube (▴: when ultraviolet is irradiated in iceberg lettuce, using a quartz tube, wherein no photo-catalyst layer of titanium dioxide is formed on its surface, as a protective tube; ▪: when ultraviolet is irradiated in iceberg lettuce, using a quartz tube, wherein a photo-catalyst layer of titanium dioxide is formed on its surface, as a protective tube; and I (error bar): indicating latent error amount related to each datum signal of data series);

FIG. 21 is a graph representing the bactericidal effect against total aerobic bacteria in ginseng over time, in the disinfecting apparatus with a lamp unit including the quartz tube on which a photo-catalyst layer of titanium dioxide is formed (▪: when ultraviolet is irradiated in ginseng, using a quartz tube, wherein a photo-catalyst layer of titanium dioxide is formed on its surface, as a protective tube; and I (error bar): indicating latent error amount related to each datum signal of data series);

FIG. 22 is a graph representing the bactericidal effect against Escherichia coli inoculated into carrot over time, in the disinfecting apparatus, depending on whether or not a photo-catalyst layer of titanium dioxide is formed on the quartz tube (♦: when carrot inoculated with E. coli is immersed in general tap water; [symbol] when ultraviolet is irradiated in carrot inoculated with E. coli, using a quartz tube, wherein no photo-catalyst layer of titanium dioxide is formed on its surface, as a protective tube; ▴: when ultraviolet is irradiated in carrot inoculated with E. coli, using a quartz tube, wherein a photo-catalyst layer of titanium dioxide is formed on its surface, as a protective tube; and I (error bar): indicating latent error amount related to each datum signal of data series);

FIG. 23 is a graph representing the bactericidal effect against Salmonella typhimurium inoculated into carrot over time, in the disinfecting apparatus, depending on whether or not a photo-catalyst layer of titanium dioxide is formed on the quartz tube (♦: when carrot inoculated with Salmonella typhimurium, is immersed in general tap water; [symbol] when ultraviolet is irradiated in carrot inoculated with Salmonella typhimurium, using a quartz tube, wherein no photo-catalyst layer of titanium dioxide is formed on its surface, as a protective tube; ▴: when ultraviolet is irradiated in carrot inoculated with Salmonella typhimurium, using a quartz tube, wherein a photo-catalyst layer of titanium dioxide is formed on its surface, as a protective tube; and I (error bar): indicating latent error amount related to each datum signal of data series);

FIG. 24 is a graph representing the bactericidal effect against Bacillus cereus inoculated into carrot over time, in the disinfecting apparatus, depending on whether or not a photo-catalyst layer of titanium dioxide is formed on the quartz tube (♦: when carrot inoculated with Bacillus cereus is immersed in general tap water; [symbol] when ultraviolet is irradiated in carrot inoculated with Bacillus cereus, using a quartz tube, wherein no photo-catalyst layer of titanium dioxide is formed on its surface, as a protective tube; ▴when ultraviolet is irradiated in carrot inoculated with Bacillus cereus, using a quartz tube, wherein a photo-catalyst layer of titanium dioxide is formed on its surface, as a protective tube; and I (error bar): indicating latent error amount related to each datum signal of data series);

FIG. 25 is a graph representing the bactericidal effect against Escherichia coli inoculated into iceberg lettuce over time, in the disinfecting apparatus, depending on whether or not a photo-catalyst layer of titanium dioxide is formed on the quartz tube (▴: when ultraviolet is irradiated in iceberg lettuce inoculated with E. coli, using a quartz tube, wherein no photo-catalyst layer of titanium dioxide is formed on its surface, as a protective tube; ▪: when ultraviolet is irradiated in iceberg lettuce inoculated with E. coli, using a quartz tube, wherein a photo-catalyst layer of titanium dioxide is formed on its surface, as a protective tube; and I (error bar): indicating latent error amount related to each datum signal of data series);

FIG. 26 is a graph representing the bactericidal effect against Listeria monocytogenes inoculated into iceberg lettuce over time, in the disinfecting apparatus, depending on whether or not a photo-catalyst layer of titanium dioxide is formed on the quartz tube (▴: when ultraviolet is irradiated in iceberg lettuce inoculated with Listeria monocytogenes, using a quartz tube, wherein no photo-catalyst layer of titanium dioxide is formed on its surface, as a protective tube; ▪: when ultraviolet is irradiated in iceberg lettuce inoculated with Listeria monocytogenes, using a quartz tube, wherein a photo-catalyst layer of titanium dioxide is formed on its surface, as a protective tube; and I (error bar): indicating latent error amount related to each datum signal of data series);

FIG. 27 is a graph representing the bactericidal effect against Salmonella typhimurium inoculated into iceberg lettuce over time, in the disinfecting apparatus, depending on whether or not a photo-catalyst layer of titanium dioxide is formed on the quartz tube (▴: when ultraviolet is irradiated in iceberg lettuce inoculated with Salmonella typhimurium, using a quartz tube, wherein no photo-catalyst layer of titanium dioxide is formed on its surface, as a protective tube; ▪: when ultraviolet is irradiated in iceberg lettuce inoculated with Salmonella typhimurium, using a quartz tube, wherein a photo-catalyst layer of titanium dioxide is formed on its surface, as a protective tube; and I (error bar): indicating latent error amount related to each datum signal of data series);

FIG. 28 is a graph representing the bactericidal effect against Bacillus subtilis spore inoculated into ginseng over time, in the disinfecting apparatus equipped with an ultraviolet lamp unit including the quartz tube on which a photo-catalyst layer of titanium dioxide is formed (▪: when ultraviolet is irradiated in ginseng inoculated with Bacillus subtilis spores, using a quartz tube, wherein a photo-catalyst layer of titanium dioxide is formed on its surface, as a protective tube; and I (error bar): indicating latent error amount related to each datum signal of data series);

FIG. 29 is a graph representing the bactericidal effect in pH 5.5 against total aerobic bacteria in broccoli over time, in the disinfecting apparatus equipped with an ultraviolet lamp unit including the quartz tube on which a photo-catalyst layer of titanium dioxide is formed (▴: when ultraviolet is irradiated in broccoli, using a quartz tube, wherein no photo-catalyst layer of titanium dioxide is formed on its surface, as a protective tube (pH 5.5); ▪: when ultraviolet is irradiated in broccoli, using a quartz tube, wherein a photo-catalyst layer of titanium dioxide is formed on its surface, as a protective tube (pH 5.5); and I (error bar): indicating latent error amount related to each datum signal of data series);

FIG. 30 is a graph representing the bactericidal effect in pH 7.5 against total aerobic bacteria in broccoli over time, in the disinfecting apparatus equipped with an ultraviolet lamp unit including the quartz tube on which a photo-catalyst layer of titanium dioxide is formed (▴: when ultraviolet is irradiated in broccoli, using a quartz tube, wherein no photo-catalyst layer of titanium dioxide is formed on its surface, as a protective tube (pH 7.5); ▪: when ultraviolet is irradiated in broccoli, using a quartz tube, wherein a photo-catalyst layer of titanium dioxide is formed on its surface, as a protective tube (pH 7.5); and I (error bar): indicating latent error amount related to each datum signal of data series);

FIG. 31 is a graph representing the bactericidal effect in pH 9.5 against total aerobic bacteria in broccoli over time, in the disinfecting apparatus equipped with an ultraviolet lamp unit including the quartz tube on which a photo-catalyst layer of titanium dioxide is formed (▴: when ultraviolet is irradiated in broccoli, using a quartz tube, wherein no photo-catalyst layer of titanium dioxide is formed on its surface, as a protective tube (pH 9.5); ▪: when ultraviolet is irradiated in broccoli, using a quartz tube, wherein a photo-catalyst layer of titanium dioxide is formed on its surface, as a protective tube (pH 9.5); and I (error bar): indicating latent error amount related to each datum signal of data series);

FIG. 32 is a graph representing the bactericidal effect in pH 5.5 against Escherichia coli inoculated into broccoli over time, in the disinfecting apparatus equipped with an ultraviolet lamp unit including the quartz tube on which a photo-catalyst layer of titanium dioxide is formed (▴: when ultraviolet is irradiated in broccoli inoculated with E. coli, using a quartz tube, wherein no photo-catalyst layer of titanium dioxide is formed on its surface, as a protective tube (pH 5.5); ▪: when ultraviolet is irradiated in broccoli inoculated with E. coli, using a quartz tube, wherein a photo-catalyst layer of titanium dioxide is formed on its surface, as a protective tube (pH 5.5); and I (error bar): indicating latent error amount related to each datum signal of data series);

FIG. 33 is a graph representing the bactericidal effect in pH 7.5 against Escherichia coli inoculated into broccoli over time, in the disinfecting apparatus equipped with an ultraviolet lamp unit including the quartz tube on which a photo-catalyst layer of titanium dioxide is formed (▴: when ultraviolet is irradiated in broccoli inoculated with E. coli, using a quartz tube, wherein no photo-catalyst layer of titanium dioxide is formed on its surface, as a protective tube (pH 7.5); ▪: when ultraviolet is irradiated in broccoli inoculated with E. coli, using a quartz tube, wherein a photo-catalyst layer of titanium dioxide is formed on its surface, as a protective tube (pH 7.5); and I (error bar): indicating latent error amount related to each datum signal of data series);

FIG. 34 is a graph representing the bactericidal effect in pH 9.5 against Escherichia coli inoculated into broccoli over time, in the disinfecting apparatus equipped with an ultraviolet lamp unit including the quartz tube on which a photo-catalyst layer of titanium dioxide is formed (▴: when ultraviolet is irradiated in broccoli inoculated with E. coli, using a quartz tube, wherein no photo-catalyst layer of titanium dioxide is formed on its surface, as a protective tube (pH 9.5); ▪: when ultraviolet is irradiated in broccoli inoculated with E. coli, using a quartz tube, wherein a photo-catalyst layer of titanium dioxide is formed on its surface, as a protective tube (pH 9.5); and I (error bar): indicating latent error amount related to each datum signal of data series);

FIG. 35 is a graph representing the bactericidal effect in pH 5.5 against Salmonella typhimurium inoculated into broccoli over time, in the disinfecting apparatus equipped with an ultraviolet lamp unit including the quartz tube on which a photo-catalyst layer of titanium dioxide is formed (▴: when ultraviolet is irradiated in broccoli inoculated with Salmonella typhimurium, using a quartz tube, wherein no photo-catalyst layer of titanium dioxide is formed on its surface, as a protective tube (pH 5.5); ▪: when ultraviolet is irradiated in broccoli inoculated with Salmonella typhimurium, using a quartz tube, wherein a photo-catalyst layer of titanium dioxide is formed on its surface, as a protective tube (pH 5.5); and I (error bar): indicating latent error amount related to each datum signal of data series);

FIG. 36 is a graph representing the bactericidal effect in pH 7.5 against Salmonella typhimurium inoculated into broccoli over time, in the disinfecting apparatus equipped with an ultraviolet lamp unit including the quartz tube on which a photo-catalyst layer of titanium dioxide is formed (▴: when ultraviolet is irradiated in broccoli inoculated with Salmonella typhimurium, using a quartz tube, wherein no photo-catalyst layer of titanium dioxide is formed on its surface, as a protective tube (pH 7.5); ▪: when ultraviolet is irradiated in broccoli inoculated with Salmonella typhimurium, using a quartz tube, wherein a photo-catalyst layer of titanium dioxide is formed on its surface, as a protective tube (pH 7.5); and I (error bar): indicating latent error amount related to each datum signal of data series);

FIG. 37 is a graph representing the bactericidal effect in pH 9.5 against Salmonella typhimurium inoculated into broccoli over time, in the disinfecting apparatus equipped with an ultraviolet lamp unit including the quartz tube on which a photo-catalyst layer of titanium dioxide is formed (▴: when ultraviolet is irradiated in broccoli inoculated with Salmonella typhimurium, using a quartz tube, wherein no photo-catalyst layer of titanium dioxide is formed on its surface, as a protective tube (pH 9.5); ▪: when ultraviolet is irradiated in broccoli inoculated with Salmonella typhimurium, using a quartz tube, wherein a photo-catalyst layer of titanium dioxide is formed on its surface, as a protective tube (pH 9.5); and I (error bar): indicating latent error amount related to each datum signal of data series);

FIG. 38 is a graph representing the growth inhibiting effect under storage against total aerobic bacteria in carrot over time, in the disinfecting apparatus equipped with an ultraviolet lamp unit including the quartz tube on which a photo-catalyst layer of titanium dioxide is formed (♦: when carrot is immersed in general tap water; [symbol] when ultraviolet is irradiated in carrot, using a quartz tube, wherein no photo-catalyst layer of titanium dioxide is formed on its surface, as a protective tube; ▴: when ultraviolet is irradiated in carrot, using a quartz tube, wherein a photo-catalyst layer of titanium dioxide is formed on its surface, as a protective tube; and I (error bar): indicating latent error amount related to each datum signal of data series); and

FIG. 39 is a graph representing the growth inhibiting effect under storage against total aerobic bacteria in iceberg lettuce over time, in the disinfecting apparatus equipped with an ultraviolet lamp unit including the quartz tube on which a photo-catalyst layer of titanium dioxide is formed ( [symbol] when iceberg lettuce is immersed in general tap water; ◯: When iceberg lettuce is immersed in chlorine water; [symbol] when ultraviolet is irradiated in iceberg lettuce, using a quartz tube, wherein no photo-catalyst layer of titanium dioxide is formed on its surface, as a protective tube; ▴: when ultraviolet is irradiated in iceberg lettuce, using a quartz tube, wherein a photo-catalyst layer of titanium dioxide is formed on its surface, as a protective tube; and I (error bar): indicating latent error amount related to each datum signal of data series).

### DETAILED DESCRIPTION

Reference will now be made in detail to the preferred embodiments of the present invention, examples of which are illustrated in the drawings attached hereinafter, wherein like reference numerals refer to like elements throughout. The embodiments are described below so as to explain the present invention by referring to the figures. The term “food” mentioned herein is referred to an object to be disinfected (for, example, fruits or vegetables).

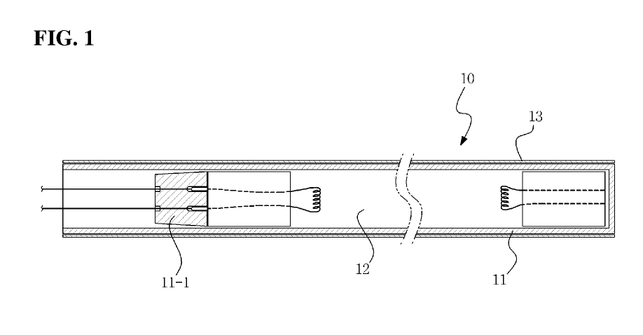

FIG. 1 is a sectional view of a UV lamp unit employed in an apparatus for disinfecting food according to a preferred embodiment of the present invention, a UV lamp unit 10 comprises a quartz tube 11 and a UV lamp 12 mounted in the quartz tube 11. A power is applied to the UV lamp 12 through a power applying unit 11-1 provided at the quartz tube 11. On the other hand, a photo-catalyst layer 13 of titanium dioxide is formed on an outer surface of the quartz tube 11.

Here, it is preferable that a photo-catalyst layer 13 of titanium dioxide formed on an outer surface of the quartz tube 11 has a thickness of 0.1 to 2.0 micrometer. Also, one end of the quartz tube 11 is hermetically sealed and the power applying unit 11-1 is provided at only the other end the quartz tube 11 for preventing water from being flowed into the quartz tube 11.

If a thickness of the photo-catalyst layer 13 of titanium dioxide is less than 0.1 micrometer, the photo-catalyst is small in quantity so that a disinfection effect is insufficient, and if a thickness of the photo-catalyst layer 13 of titanium dioxide is more than 2.0 micrometer, a transmittance of ultraviolet ray is low so that a disinfection effect obtained by the photo-catalyst layer of titanium dioxide is insufficient.

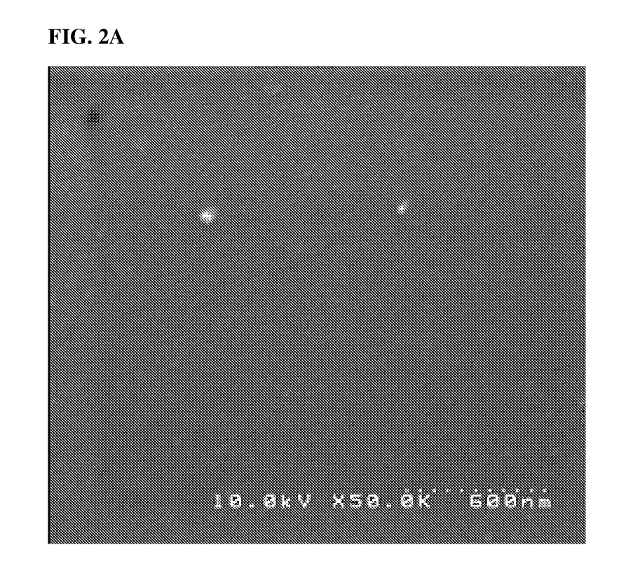

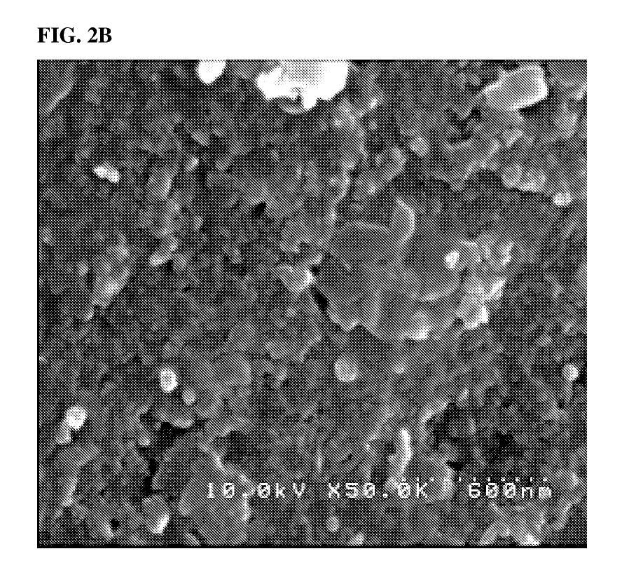

FIG. 2A is a scanning electron microscopy photograph of a quartz tube on which a photo-catalyst layer of titanium dioxide is not formed and FIG. 2B is a scanning electron microscopy photograph of a quartz tube having a photo-catalyst layer of titanium dioxide formed on an outer surface thereof.

In FIG. 2B, titanium dioxide particle constituting the photo-catalyst layer of titanium dioxide has a nano size of 10 to 60 nm and has a thickness of 700 to 900 nm.

A plurality of UV lamp units 10 having the structure shown in FIG. 1 are mounted in the apparatus for disinfecting food according to the present invention.

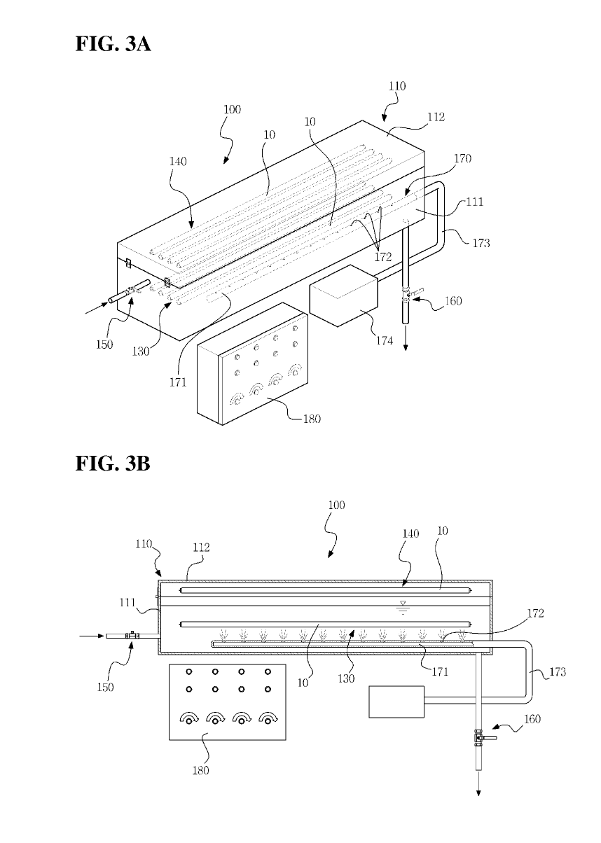

FIG. 3A and FIG. 3B are perspective view and front view, respectively, of a batch type apparatus for disinfecting food according to a first embodiment of the present invention, the apparatus 100 for disinfecting food according to this embodiment comprises a bath 110 which includes a case 111 and a cover 112 provided openable/closably at the case 111.

The bath 110 is provided with a water-supplying unit 150 for supplying water into the case 111 and a water-draining unit 160 for draining water in the case 111 to an exterior.

The apparatus 100 for disinfecting food according to the first embodiment of the present invention further comprises a first UV lamp unit assembly 130 mounted in the case 111 of the bath 110. The first UV lamp unit assembly 130 includes at least one UV lamp unit 10, and the UV lamp unit 10 has the structure which is the same as that of the UV lamp unit shown in FIG. 1.

While the first UV lamp unit assembly 130 is shown to be disposed at a lower portion of the case 111 in FIG. 3A and FIG. 3B, the location of the first UV lamp unit assembly 130 in the case 111 is not limited thereto. In the UV lamp unit 10, on the other hand, the quartz tube 11 acts as a protective tube of the UV lamp 12, and so the UV lamp 12 is not contacted directly with water in the case 111.

In the apparatus 100 for disinfecting food having the structure as described above, an object food is disinfected and washed by the UV lamp 12 provided in the quartz tube 11 having the photo-catalyst layer 13 of titanium dioxide formed on an outer surface thereof.

That is, the case 111 of the bath 110 is filled with water supplied through the water-supplying unit 150 and the food to be disinfected is immersed in water. If the power is then applied to the first UV lamp unit assembly 130 placed below food, the UV lamp units 10 emit the ultraviolet ray and the photo-catalyst layer 13 of titanium dioxide formed on an outer surface of the quartz tube 11 constituting each UV lamp unit 10 is activated so that the food in the case 111 can be steeped and disinfected.

After performing the disinfection process as described above, water in the case 111 is drained to an exterior through the water-draining unit 160.

On the other hand, the apparatus 100 for disinfecting food may further comprise an air-blowing means 170. The air-blowing means 170 includes an air flow line 171 provided below the first UV lamp unit assembly 130, an air supplying unit 174 installed outside the case 111 and a connecting line 173 connecting the air flow line 171 and the air supplying unit 174. Here, a plurality of openings 172 are formed on the air flow line 171.

Air supplied from the air supplying unit 174 is supplied to the air flow line 171 through the connecting line 173, and so air is injected in water through the openings 172 formed on the air flow line 171.

Due to air injected in water as described above, food is moved in water so that ultraviolet ray is uniformly and sufficiently radiated onto food and the activated photo-catalyst layer 13 of titanium dioxide promotes a disinfection function of the ultraviolet ray.

In addition, the apparatus 100 for disinfecting food according to this embodiment may further comprise a second UV lamp unit assembly 140 mounted on an inner surface of the cover 112 of the bath 110. The second UV lamp unit assembly 140 includes of at least one UV lamp unit 10, and the UV lamp unit 10 has the structure which is the same as that of the UV lamp unit shown in FIG. 1.

Ultraviolet ray emitted from the first UV lamp unit assembly 130 provided at a low portion of the case 111 may not be radiated sufficiently onto all food, in particular, where an object food is placed at an upper region of the case 111 and the activated photo-catalyst layer 13 of titanium dioxide may hardly promote a disinfection function of the ultraviolet ray.

To make up for the above weak points, in this embodiment, the second UV lamp unit assembly 140 is mounted on an inner surface of the cover 112. Ultraviolet ray emitted from the second UV lamp unit assembly 140 can be radiated onto food placed at an upper region of the case 111 so that all food in the case 111 can be disinfected completely.

In the above disinfection process, on the other hand, ultraviolet ray emitted from the first and second UV lamp unit assemblies 130 and 140 is not leaked out of the case 111 by the cover 112 provided at the case 111.

Reference numeral “180” in FIG. 3A and FIG. 3b indicates a control box for controlling the power to be applied to the first and second UV lamp unit assemblies 130 and 140 and the air supplying unit 174.

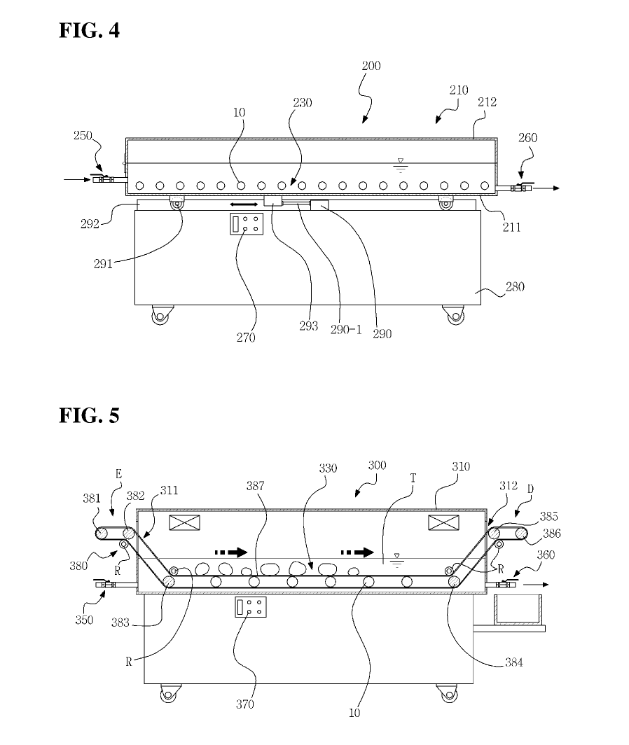

FIG. 4 is a view illustrating a batch type apparatus for disinfecting food according to a second embodiment of the present invention, the apparatus 200 for disinfecting food according to this embodiment comprises a bath 210 including a case 211 and a cover 212 provided openable/closably at the case 211.

The bath 210 is provided with a water-supplying unit 250 for supplying water into the case 211 and a water-draining unit 260 for draining water in the case 211 to an exterior.

The apparatus 200 for disinfecting food according to this embodiment of the present invention further comprises a UV lamp unit assembly 230 mounted in the case 211 of the bath 210. The UV lamp unit assembly 230 includes at least one UV lamp unit 10, and the UV lamp unit 10 has the structure which is the same as that of the UV lamp unit shown in FIG. 1.

Contrary to FIG. 3A and FIG. 3B showing that the UV lamp units 10 are disposed in a longitudinal direction of the case 111, the UV lamp units 10 in the apparatus 200 are disposed in a transverse direction of the case 211. However, the location of the UV lamp units is not limited thereto.

It is preferable that the UV lamp unit assembly 230 is disposed at a lower portion of the case 211 as shown in FIG. 4. But the location of the UV lamp unit assembly 230 in the case 211 is not limited thereto. In the UV lamp unit 10, on the other hand, the quartz tube 11 acts as a protective tube of the UV lamp 12, and so the UV lamp 12 is not contacted directly with water in the case 211.

Dsinfection process and function in the apparatus 200 according to the second embodiment of the present invention utilizing the UV lamp unit assembly 230 are same as those in the apparatus 100 according to the first embodiment, and so the detail description thereon is omitted.

The apparatus 200 for disinfecting food according to the second embodiment further comprises a base 280, and the bath 210 can be reciprocated within a certain distance on the base 280.

That is, a plurality of rollers 291 are rotatably mounted on a lower surface of the case 211 and a guide channel 292 in which the rollers 291 are received is formed on an upper surface of the base 280.

In addition, a fixture 293 having a certain height is provided on a lower surface of the case 211 and a driving means 290 (for example, a shaking motor or air/hydraulic cylinder) for reciprocating the case 211 linearly is mounted on the base 280. A free end of an operation arm 290-1 of the driving means 290 is fixed to the fixture 293 of the case 211.

According to an operation of the driving means 290, the operation arm 290-1 is reciprocated from right to left (in an arrow direction in FIG. 4) so that the case 211 to which the operation arm 290-1 is fixed can be reciprocated on the base 280.

At this time, since the rollers 291 mounted on the case 211 are received in the guide channel 292 fixed on the upper surface of the base 280, the case 211 may reciprocate linearly on the base 280 along the guide channel 292.

By the reciprocation of the case 211 on the base 280 as described above, a vibration is exerted to a food immersed in water in the case so that the food can be more effectively washed by water.

Reference numeral “270” in FIG. 4 indicates a control box for controlling the power to be applied to the UV lamp unit assembly 230 and the driving means 290 for reciprocating the case.

FIG. 5 is a view illustrating a continuous type apparatus for disinfecting food according to a third embodiment of the present invention.

The apparatus 300 comprises a bath 310 to which a cover is not mounted. The bath 310 is provided with a water-supplying unit 350 for supplying water into the bath 310 and a water-draining unit 360 for draining water in the bath 310 to an exterior.

The apparatus 300 further comprises a UV lamp unit assembly 330 mounted in the bath 310. The UV lamp unit assembly 330 includes at least one UV lamp unit 10, and the UV lamp unit 10 has the structure which is the same as that of the UV lamp unit shown in FIG. 1.

It is preferable that the UV lamp unit assembly 330 is disposed at a lower portion of the bath 310 as shown in FIG. 5. But the location of the UV lamp unit assembly 330 in the bath 310 is not limited thereto. In the UV lamp unit, on the other hand, the quartz tube 11 acts as a protective tube of the UV lamp 12, and so the UV lamp 12 is not contacted directly with water in the bath 310.

Disinfection process and function in the apparatus 300 according to the third embodiment utilizing the UV lamp unit assembly 330 are same as those in the apparatuses 100 and 200 according to the first and second embodiments, and so the detail description thereon is omitted.

The apparatus 300 further comprises a conveyer device 380 provided in the bath 310.

The conveyer device 380 comprises a driving roller 381, a plurality of driven rollers 382, 383, 384, 385 and 386 and a conveyer belt 387 wounded around the rollers 381, 382, 383, 384, 385 and 386. As shown in FIG. 5, the UV lamp unit assembly 330 is disposed below the conveyer device 380, more preferably, in a space between an upper portion and a lower portion of the conveyer belt 387.

The conveyer device 380 is divided into an inlet portion E formed at a first outside of the bath 310, a conveying portion T formed in the bath 310 and a discharging portion D formed at a second outside of the bath 310. Here, first and second openings 311 and 312 are formed on first and second walls of the bath 310, respectively, for arranging the conveyer belt 387 and conveying food placed on the conveyer belt 387.

The inlet portion E formed horizontally at the first outside of the bath 310 includes at least two rollers 381 and 382, and the discharging portion D formed at horizontally at the second outside of the bath 310 also includes at least two rollers 385, 386. And, the conveying portion T is formed by at least two rollers 383 and 384 disposed in the bath 310.

As shown in FIG. 5, one conveyer belt 387 is wounded around all the rollers 381, 382, 383, 384, 385 and 386.

On the other hand, the bath 310 is filled with water, and the conveying portion T of the conveyer device 380 should be immersed in water. Also, the inlet portion E and the discharging portion D of the conveyer device 380 (that is, the openings 311, 312 formed on the side walls of the bath 310) should be disposed at positions higher than a surface of water for preventing water from being leaked through the openings 311, 312. That is, there is a height difference between the inlet portion E (the discharging portion D) and the conveying portion T of the conveyer device 380 as shown in FIG. 5.

Accordingly, food putted on the conveyer belt 387 corresponding to the inlet portion E of the conveyer device 380 is conveyed into the bath 310 by the transferring conveyer belt 387 through the first opening 311 formed on the first side wall of the bath 310.

Food putted on the conveyer belt 387 and reaching the conveying portion T is immersed in water in the bath 310, and a disinfection treatment is then performed by the ultraviolet ray emitted from the UV lamp unit assembly 330 during food is conveyed in the bath 310 by the conveyer belt 387. The food, which has been subject to the disinfection treatment, is discharged out of the bath 310 by the conveyer belt 387 through the second opening 312 formed on the second wall of the bath 310.

In the apparatus 300, food is fed continuously to the conveyer device 380, and so food can be conveyed continuously in water in the bath 310 by the conveyer belt 387 to perform continuously the disinfection treatment for food.

The structure and function of the conveyer belt 387 are detailed later. Also, reference numeral “370” in FIG. 5 indicates a control box for controlling the power to be applied to the UV lamp unit assembly 330 and a driving unit (not shown) means providing a driving force to the driving roller 381 of the conveyer device 380.

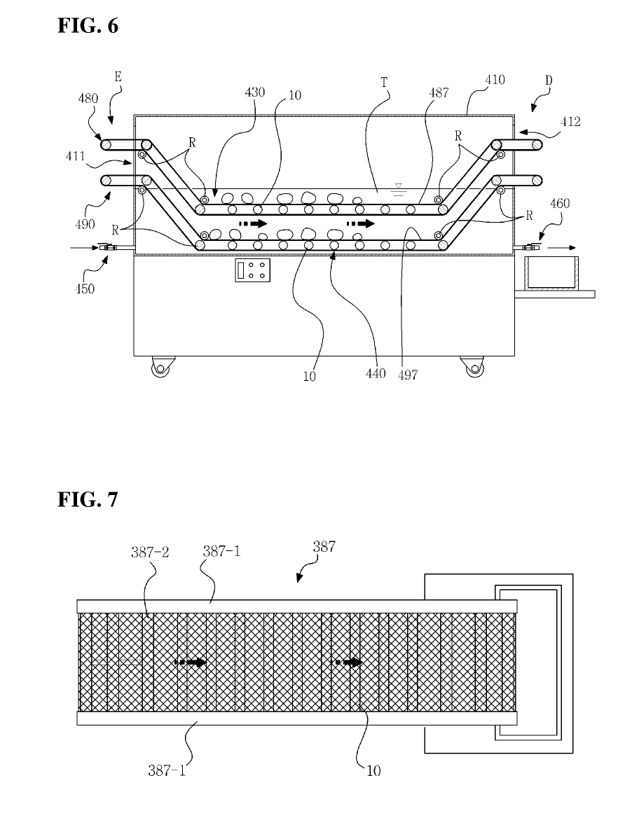

FIG. 6 is a view illustrating another continuous type apparatus for disinfecting food according to a fourth embodiment of the present invention.

The apparatus 400 comprises a bath 410 to which a cover is not mounted.

The bath 410 is provided with a water-supplying unit 450 for supplying water into the bath 410 and a water-draining unit 460 for draining water in the bath 410 to an exterior.

The apparatus 400 comprises first and second UV lamp unit assemblies 430 and 440 mounted in the bath 410. Each of the first and second UV lamp unit assemblies 430, 440 includes at least one UV lamp unit 10, and the UV lamp unit 10 has the structure which is the same as that of the UV lamp unit shown in FIG. 1.

In the bath 410, as shown in FIG. 6, the first and second UV lamp unit assemblies 430 and 440 are spaced from each other at a certain interval.

Disinfection process and function in the apparatus 400 according to this embodiment are same as those in the apparatuses 100 and 200 according to the first and second embodiments, and so the detail description thereon is omitted.

The apparatus 400 further comprises a first conveyer device 480 provided in the bath 410 and a second conveyer device 490 provided below the first conveyer device 480.

Structural members and relation between the structural members of each of the first and second conveyer devices 480 and 490 are the same as those of the conveyer device 380 of the apparatus 300 according to the third embodiment, and so the detail description thereon is omitted.

As shown in FIG. 6, the first UV lamp unit assembly 430 is disposed in a space between an upper portion and a lower portion of the conveyer belt 487 of the first conveyer device 480, and the second UV lamp unit assembly 440 is disposed in a space between an upper portion and a lower portion of the conveyer belt 497 of the second conveyer device 490.

Accordingly, a disinfection treatment for food conveyed by the first conveyer device 480 in the bath 410 is performed by the first UV lamp unit assembly 430, and a disinfection treatment for food conveyed by the second conveyer device 490 in the bath 410 is performed by the second UV lamp unit assembly 440. Accordingly, as compared with the apparatus 300, the apparatus 400 can perform a disinfection treatment for larger amount of food per unit time.

Here, first and second openings 411 and 412 are formed on first and second walls of the bath 410, respectively, for arranging the conveyer belts 487 and 497 and conveying food placed on the conveyer belts 487 and 497.

On the other hand, the bath 410 is filled with water, and so the conveying portions T of the first and second conveyer devices 480 and 490 should be immersed in water. Also, the inlet portions E and the discharging portions D of the first and second conveyer devices 480 and 490 (that is, the openings 411, 412 formed on the side walls of the bath 410) should be disposed at positions higher than a surface of water for preventing water from being leaked through the openings 411, 412.

The structure and function of the conveyer belts 487 and 497 are detailed later. Also, reference numeral “470” in FIG. 6, which is not illustrated, indicates a control box for controlling the power to be applied to the first and second UV lamp unit assemblies 430 and 440 and a driving unit (not shown) means providing a driving force to the driving rollers of the first and second conveyer devices 480 and 490.

The conveyer belts 387, 487 and 497 constituting the conveyer devices 380, 480 and 490 of the apparatuses for disinfecting food according to the third and fourth embodiments have the same structures and functions. Accordingly, the structure and function of the conveyer belt 387 shown in FIG. 6 are illustrated as an example.

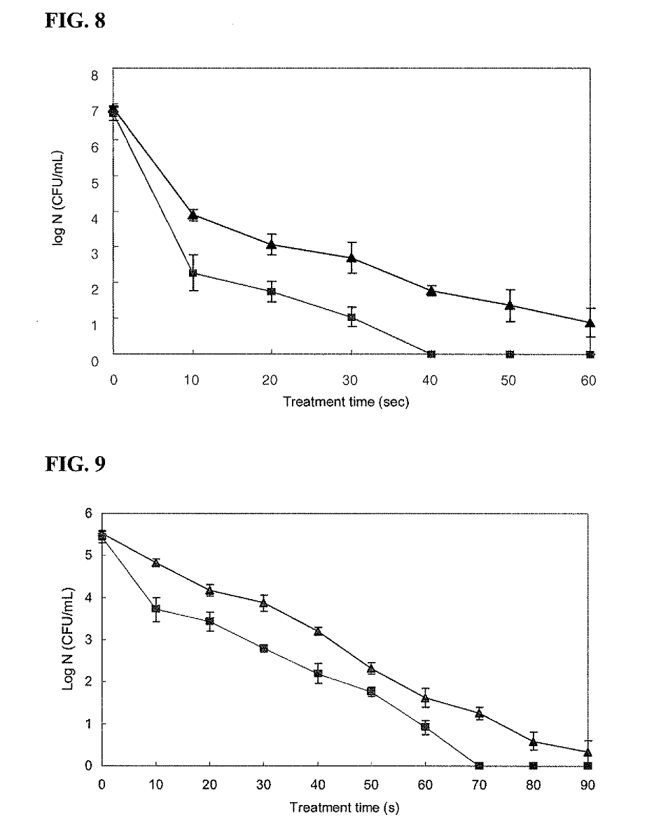

FIG. 7 is a plane view of the conveyer belt 387 of the conveyer device 380 shown in FIG. 5. The loop shaped conveyer belt 387 comprises a pair of supporting bodies 387-1 spaced apart from each other and a mesh sheet 387-2 secured to the supporting bodies 387-1.

The supporting bodies 387-1 are the member being in contact with all the rollers the 381, 382, 383, 384, 385 and 386. In particular, the supporting bodies 387-1 are moved (rotated) by the rotational force transmitted from the driving roller (for example, 381). Accordingly, it is preferable that the supporting bodies 387-1 are made from material having an excellent friction resistance, for example, an elastic material, for receiving a rotational force from the driving roller 381 without generating slip and contacting with the rollers 382, 383, 384, 385 and 386.

On the other hand, the UV lamp unit assembly 330 is disposed between a upper portion and a lower portion of the conveyer belt 387 in the bath 310, and so the ultraviolet ray emitted from the UV lamp unit assembly 330 should be passed through the upper portion of the conveyer belt 387 and then radiated to food putted on and conveyed by the conveyer belt 387.

In the conveyer belt 387, a region on which food is putted is formed of the mesh sheet 387-2, and so the ultraviolet ray emitted from the UV lamp unit assembly 330 can be passed through the mesh sheet 387-2 (that is, an upper portion of the conveyer belt 387) and then radiated to food. Accordingly, a maximum effect of the disinfection treatment for food can be obtained.

Here, due to the structure of the conveyer belt 387, the UV lamp unit assembly 330 may be disposed below the conveyer belt 387, not in a space between the upper portion and the lower portion of the conveyer belt 387.

As shown in FIG. 5, on the other hand, in order to prevent water from being leaked through the openings 311 and 312 formed on the bath 310, the inlet portion E and the discharging portion D of the conveyer device 380 should be disposed at positions higher than a surface of water in the bath 310. Accordingly, a portion of the conveyer belt 380 connecting the inlet portion E and the conveying portion T and a portion connecting the discharging portion D and the conveying portion T become inclined portions.

In this structure, when food is reached the above inclined portions of the conveyer belt 387, food may be deviated from the conveyer belt 387. In order to prevent the above problem, it is possible to fix a plurality of pins having a certain height to an outer surface of the mesh sheet 387-2 of the belt 387 to hold food on the conveyer belt.

On the other hand, reference numeral “R” in FIG. 5 and FIG. 6 indicates idle rollers to provide for supporting the conveyer belts 387, 487 and 497. The idle rollers R are mounted rotatably on walls of the bath (or case) and support the supporting bodies 387-1 (in FIG. 7) of the conveyer belts 387, 487 and 497 so that a conveyance of food is not influenced by the idle rollers R.

In the batch or continuous type disinfecting apparatus, according to the present invention, using the ultraviolet lamp unit equipped with an ultraviolet lamp in the quartz tube on which a photo-catalyst of titanium dioxide is formed, as explained above, a non-thermal disinfecting method is adapted for securing stability of foods that a heat treatment is impossible, and a physical method is employed rather than a chemical method.

The photo-catalyst disinfecting apparatus of the present invention may be utilized in the food industry, and may be effectively employed in a pre-treatment step, for example, a washing step and the like, in which microorganisms are reduced for maintaining food freshness and extending food shelf life after harvesting foods that the heat treatment is impossible such as vegetable, fruit, cereals or egg used in eating in the raw or green juices.

Using ultraviolet having strong bactericidal capacity and a photo-catalyst of titanium dioxide, pathogenic microorganisms, virus, fungi, and the like can be not only sterilized in a short time, but also organic synthetic agrichemicals remained in surfaces of foods can be degraded by organic degradation ability of the photo-catalyst. There is an advantage that by simultaneously sterilizing foods in a short time and removing the remained agrichemicals, harmless foods may be circulated.

The present invention is explained in more detail through the following examples. These examples are intended for particularly explaining the present invention, but the scope of the present invention is not limited to these examples.

### EXAMPLE 1

### Surface of Quartz Tube on Which the Photo-Catalyst Layer of Titanium Dioxide is Formed

The quartz tube, used in the present invention, having the photo-catalyst layer of titanium dioxide formed on the outer surface thereof was observed with a scanning electron microscopy, compared with the quartz tube on which no photo-catalyst layer of titanium dioxide was formed. The results are represented in FIGS. 2A and 2B.

It could be noted that the quartz tube, on which no photo-catalyst layer of titanium dioxide was formed, had a smooth surface, as shown in FIG. 2A, while the quartz tube, on which the photo-catalyst layer of titanium dioxide was formed, had a surface on which a thin layer of titanium dioxide was formed, as shown in FIG. 2B. It was confirmed that the titanium dioxide particles had a size of 10 to 60 nm and were fixed with a thickness of 700 to 900 nm.

### EXAMPLE 2

### Photo-Catalyst Disinfecting Apparatus

Using the disinfecting apparatus as shown in FIG. 3 among various types of disinfecting apparatuses, five (5) quartz tubes with outer diameter×inner diameter×length of 25×22×900 mm on which the thin layer of titanium dioxide was formed so as to maintain high UV transmittance, were placed inside the cover and five (5) quartz tubes with outer diameter×inner diameter×length of 25×22×1090 mm were placed at the bottom of inside space in the case.

In addition, the UV lamp being irradiated with an intensity of 250 W/m² at 254 nm was equipped inside each quartz tube to carry out an experiment about the bactericidal effect of disinfecting apparatus. To prove the superiority of bactericidal effect by the photo-catalyst, a control group was experimented using the quartz tube on which no thin layer of titanium dioxide was formed as a protecting tube of the UV lamp unit under the other conditions same as those of the experimental group.

### EXAMPLE 3

### Bactericidal Effect of Photo-Catalyst Disinfecting Apparatus Against Bacteria

Using the disinfecting apparatus according to the present invention, the bactericidal effect was experimented against Escherichia coli (ATCC 25922), Listeria monocytogenes (ATCC 15313), Bacillus cereus (ATCC 11778). Salmonella typhimurium (ATCC 14028), and Bacillus subtilis (ATCC 11774) as representative harmful bacteria problematic in fresh foods.

Escherichia coli was cultured in a lactose broth (Difco, USA) under 150 rpm shaking incubator at 35° C. for 24 hours, Listeria monocytogenes was cultured in a nutrient broth (Difco, USA) under 200 rpm shaking incubator at 36° C. for 24 hours and Bacillus cereus was cultured in a brain heart infusion (Difco, USA) under 150 rpm shaking incubator at 30° C. for 24 hours, Salmonella typhimurium was cultured in a tryptic soy broth (Difco, USA) under 150 rpm shaking incubator at 35° C. for 24 hours, and Bacillus subtilis was first cultured in a Nutrient Broth (Difco, USA) at 30° C. for 24 hours and then cultured in a Nutrient Agar comprising 0.05% MnSO₄ at 30° C. for 10 days, and only spores were separated therefrom with ultrasonic irradiation and centrifugation to culture them. All bacteria were second cultured, were set up to have the same concentrations in the photo-catalyst disinfecting apparatus and were experimented.

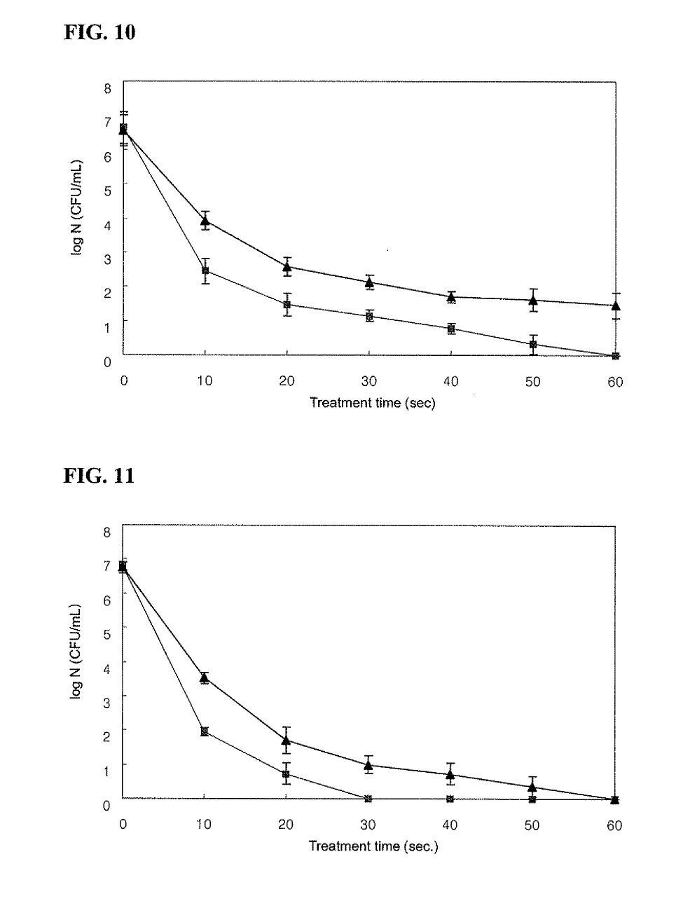

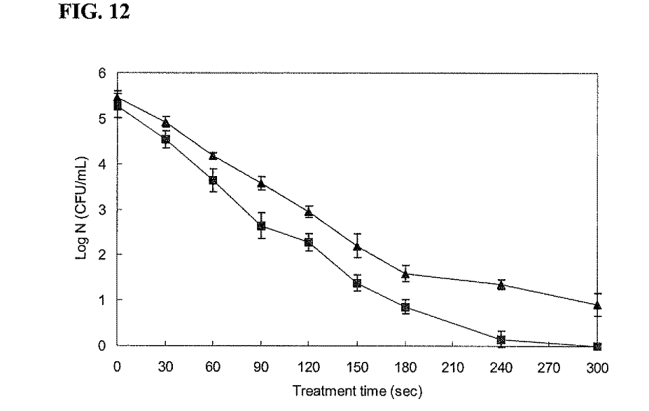

Samples were collected after photo-catalytic reaction at certain time interval and then Bacteria count was measured by a standard plate count method using Eosin methylene blue agar (Difco, USA) for Escherichia coli, Bacillus cereus agar base (Oxoid, England) that an egg-yolk and Bacillus cereus-selective supplement were added for Bacillus cereus, a nutrient agar for Listeria monocytogenes and Bacillus subtilis, and deoxycholate citrate agar (Difco, USA) for Salmonella typhimurium. The experimental results are represented in FIGS. 8 to 12.

As shown in FIGS. 8 to 12, the time required for complete sterilization by the disinfecting apparatus according to the present invention was 40 seconds in case of Escherichia coli, 70 seconds in case of Listeria monocytogenes, 60 seconds in case of Bacillus cereus, 30 seconds in case of Salmonella typhimurium, and 4 minutes in case of Bacillus subtilis spores, whereas on conventional single ultraviolet treatment, longer time was required or complete sterilization was not achieved even for 4 minutes.

Accordingly, it could be noted through the present experimental example that when the photo-catalyst disinfecting method of the present invention was applied to any process for disinfecting and treating harmful microorganisms, the effect was very good.

### EXAMPLE 4

### Change of Outer Shape of Microorganisms by Photo-Catalytic Reaction

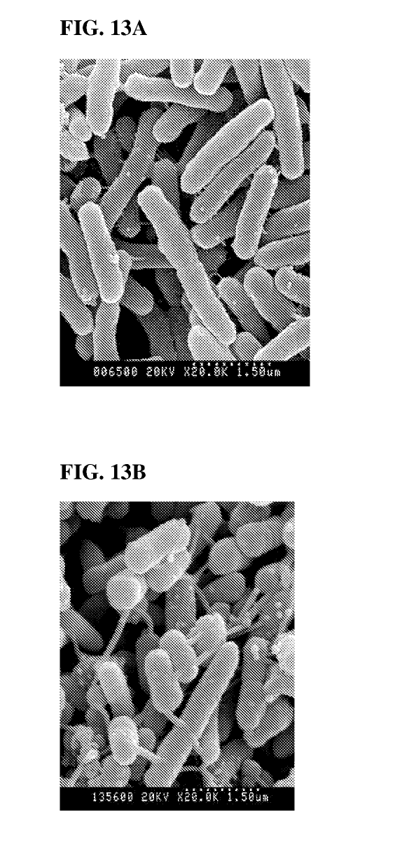

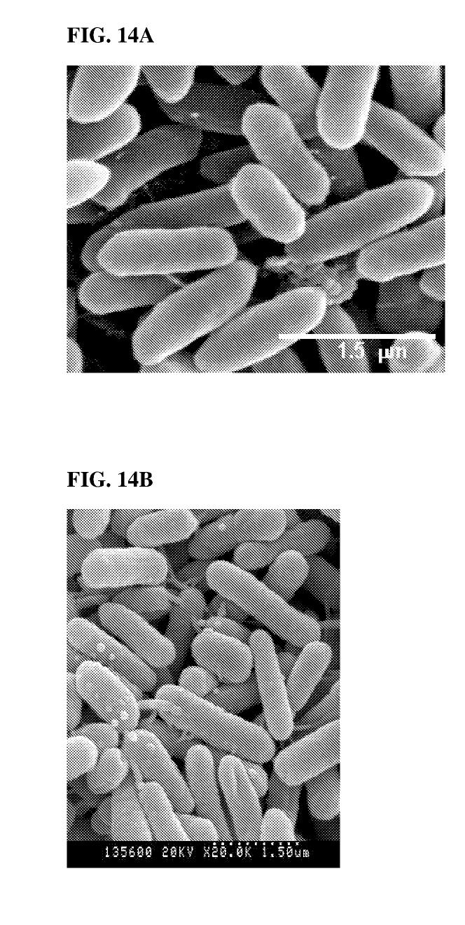

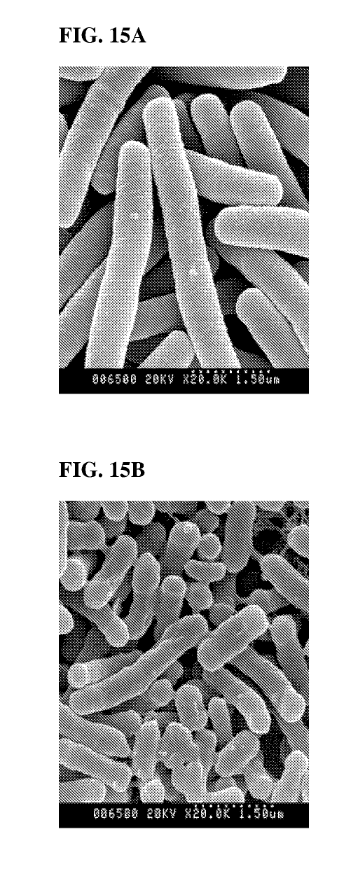

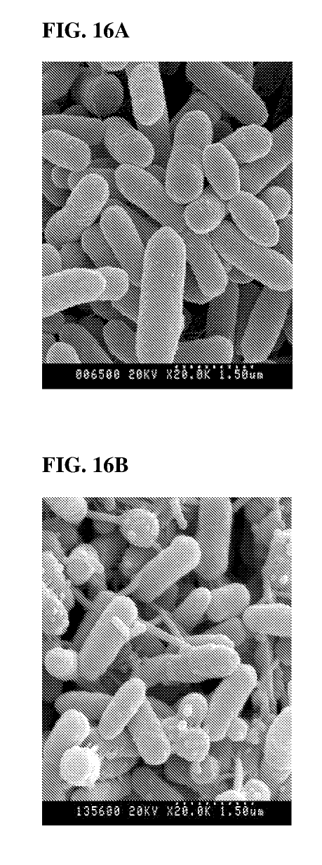

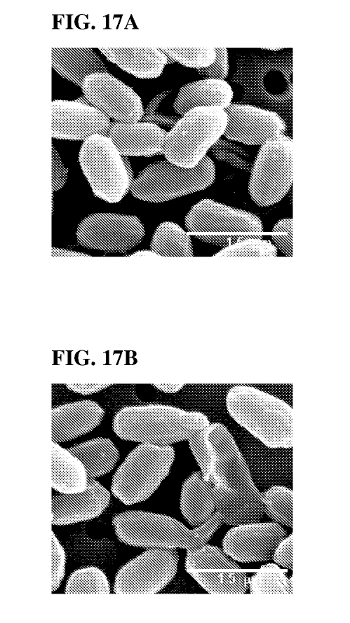

The change of outer shapes in the representative harmful bacteria according to Example 3 above, E. coli, Listeria monocytogenes, Bacillus cereus, Salmonella typhimurium, and Bacillus subtilis spores, after photo-catalytic reaction were observed with a scanning electron microscopy and the results were represented in FIGS. 13 to 17.

As shown in FIGS. 13 to 17, the outer shape of bacteria treated with only ultraviolet for 1 minute had an appearance of rod with certain shape of slow curve (see FIGS. 13A, 14A, 15A, 16A and 17A). But, in case of carrying out together with photo-catalyst treatment for 1 minute, cell surfaces were torn or caved in and thus the outer shape was uneven (see FIGS. 13B, 14B, 15B, 16B and 17B). Therefore, it could be identified that the photo-catalyst treatment gave bacterial cell surfaces severe damage.

### EXAMPLE 5

### Bactericidal Effect of Photo-Catalyst Disinfecting Apparatus Against Bacteria Present in Food Raw Materials

After purchasing raw materials of representative fresh foods under sale and circulation on the market, carrot, Angelica keiskei, iceberg lettuce, and ginseng used in raw materials of health foods, the bactericidal effect of photo-catalyst disinfecting apparatus was identified against total aerobic bacteria practically present in foods.

To measure the bactericidal effect against total aerobic bacteria present in carrot and Angelica keiskei, 4 kg of carrot and Angelica keiskei cut with certain size each were dipped into the photo-catalyst disinfecting apparatus and then the change of total aerobic bacteria count was measured over time. In case of iceberg lettuce and ginseng, they were cut with certain size and then were dipped into the disinfecting apparatus according to the present invention by 2 kg to treat them.

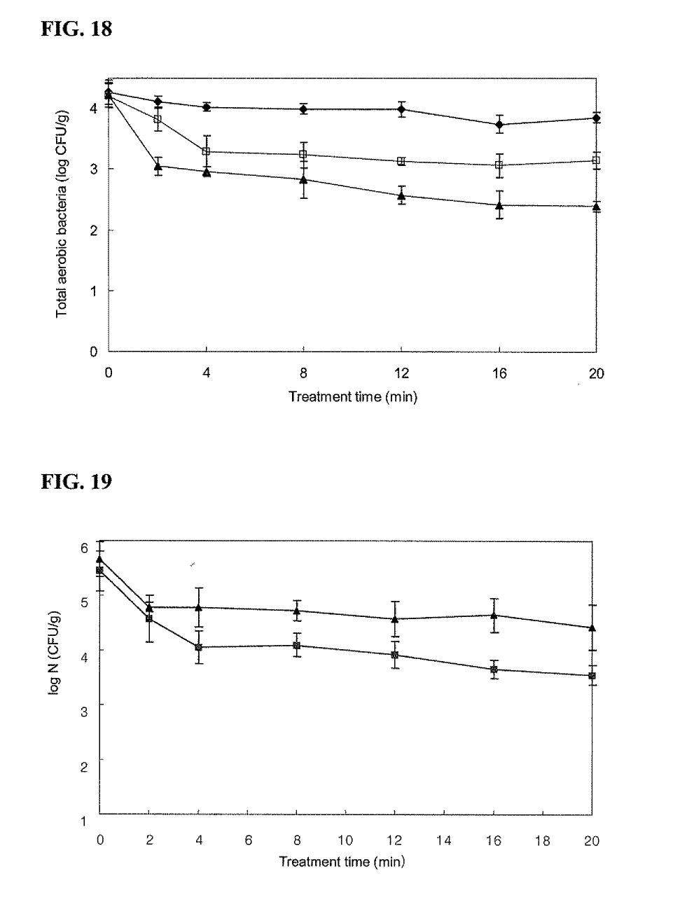

Samples were weighed, placed in a stomacher plastic bag (Nasco Whirl-pak filter bag, USA), diluted ten (10) times, and homogenized for 120 seconds using a stomacher (AES MIX-2, France). Then, a liquid phase portion was taken and diluted step by step to inoculate the specimen into a dry layer medium for total aerobic bacteria, Petrifilm™ aerobic count plate (PAC; 3M, USA), and culture it at 35° C. for 24 hours. Then, total aerobic bacteria number was counted to measure the change of bacteria count according to the reaction time of the photo-catalyst apparatus. The experimental results are represented in FIGS. 18 to 21.

As shown in FIG. 18, when it was treated with only ultraviolet in case of carrot, total aerobic bacteria count was reduced by 1.0 log CFU (colony forming unit)/g after 4 minutes and then no more bactericidal effect was represented. On the contrary, when it was carried out together with the photo-catalyst treatment, it was reduced by 1.4 log CFU/g after 4 minutes and after 20 minutes, the reducing effect of 1.8 log CFU/g was represented. That is, it could be noted that in only 20 minutes, about 99% of bacteria was reduced.

As shown in FIG. 19, when it was treated with only ultraviolet in case of Angelica keiskei, total aerobic bacteria count was reduced by 0.9 log CFU/g after 4 minutes and reduced by 1.2 log CFU/g after 20 minutes. However, when it was carried out together with the photo-catalyst treatment, it was reduced by 1.4 log CFU/g after 4 minutes and after 20 minutes, the reducing effect of 1.9 log CFU/g was represented. That is, it could be noted that in only 20 minutes, about 99% of bacteria was reduced.

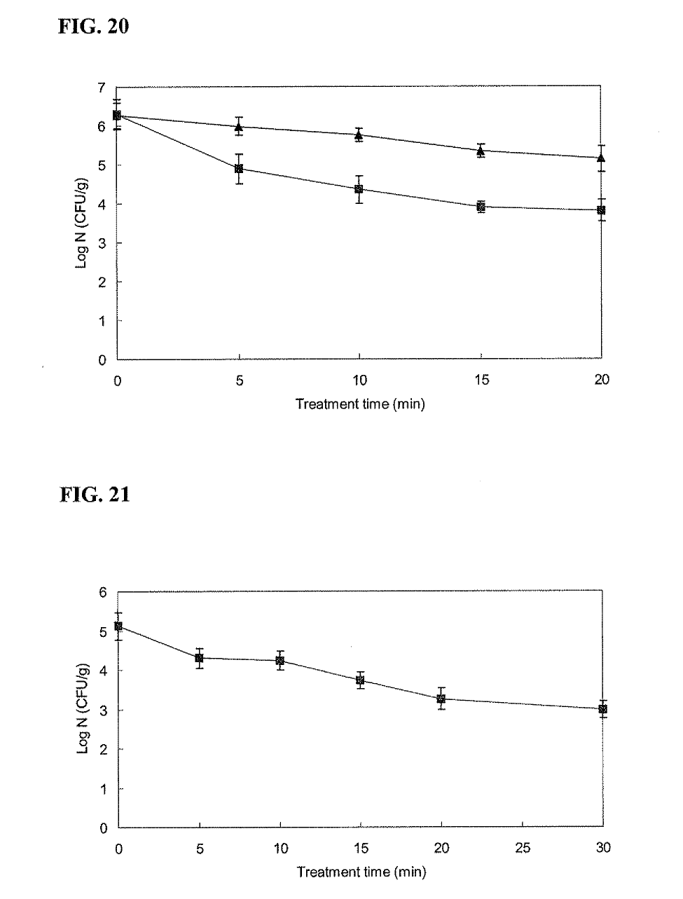

As shown in FIG. 20, when it was treated with only ultraviolet in case of iceberg lettuce, total aerobic bacteria count was reduced by 0.3 log CFU/g in only 5 minutes and reduced by 0.9 log CFU/g after 20 minutes. However, when it was carried out together with the photo-catalyst treatment, it was reduced by 1.0 log CFU/g after 5 minutes and reduced by 1.8 log CFU/g after 20 minutes. That is, it could be noted that in only 20 minutes, about 99% of bacteria was reduced.

As shown in FIG. 21, in case of ginseng, it was reduced by 0.8 log CFU/g in 5 minutes by treating it with the photo-catalyst and reduced by 2.1 log CFU/g after 30 minutes. That is, it could be noted that in only 30 minutes, about 99.9% of bacteria was reduced.

It could be identified through the present experimental example that when the photo-catalyst disinfecting method was applied to a pre-treatment step, for example, a washing step, and the like, in which microorganisms were reduced for maintaining food freshness and extending food shelf life after harvesting fresh foods that the heat treatment was impossible, the effect was very good.

### EXAMPLE 6

### Bactericidal Effect of Photo-Catalyst Disinfecting Apparatus Against Bacteria Inoculated into Foods

Harmful microorganisms were inoculated into representative fresh food raw materials circulated on the market, carrot, iceberg lettuce, and health food raw materials, ginseng, and the bactericidal effect of the photo-catalyst disinfecting apparatus was identified. To measure the bactericidal effect against Escherichia coli, Salmonella typhimurium, and Bacillus cereus inoculated into carrot and Escherichia coli, Listeria monocytogenes, and Salmonella typhimurium inoculated into iceberg lettuce, 2 kg of carrot and iceberg lettuce cut with certain size each were sterilized through ultraviolet irradiation for 30 minutes and were immersed into each bacteria solution. Then, these solutions were dipped into the photo-catalyst disinfecting apparatus and then the change of harmful bacteria count was measured over time.

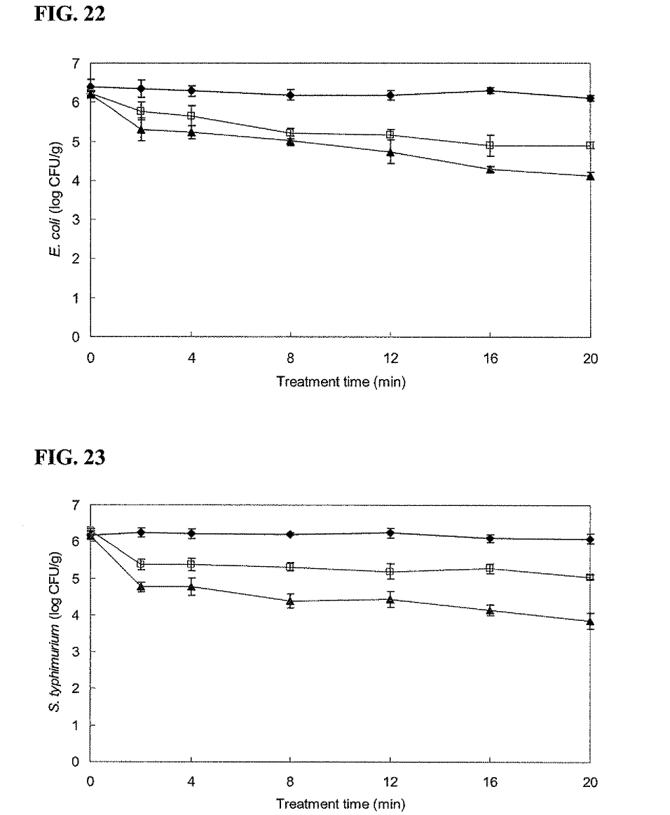

In case of ginseng, it was cut with certain size, was inoculated by applying Bacillus subtilis spore solution on 2 kg of ginseng and then was dipped into the disinfecting apparatus according to the present invention to treat them. Samples were weighed, placed in a stomacher plastic bag (Nasco Whirl-pak filter bag, USA), diluted ten (10 ) times, and homogenized for 120 seconds using a stomacher (AES MIX-2 , France). Then, a liquid phase portion was taken and diluted step by step to inoculate the specimen into the same medium as Example 4 above by standard plate count method, and culture it at 35° C. for 24 hours. Then, bacteria number was counted to measure the change of bacteria count according to the reaction time of the photo-catalyst apparatus. The experimental results are represented in FIGS. 22 to 28.

As shown in FIG. 22, when it was treated with only ultraviolet in case of E. coli inoculated into carrot, the bacteria were reduced by 0.5 log CFU/g after 4 minutes and reduced by 1.3 log CFU/g after 20 minutes. However, when it was carried out together with the photo-catalyst treatment, it was reduced by 1.2 log CFU/g after 4 minutes and after 20 minutes, the reducing effect of 2.1 log CFU/g was represented. That is, it could be noted that in only 20 minutes, about 99.9 % of bacteria was reduced.

As shown in FIG. 23, when it was treated with only ultraviolet in case of Salmonella typhimurium inoculated into carrot, the bacteria were reduced by 0.6 log CFU/g after 4 minutes and reduced by 1.2 log CFU/g after 20 minutes. However, when it was carried out together with the photo-catalyst treatment, it was reduced by 1.4 log CFU/g after 4 minutes and after 20 minutes, the reducing effect of 2.3 log CFU/g was represented. That is, it could be noted that in only 20 minutes, about 99.9% of bacteria was reduced.

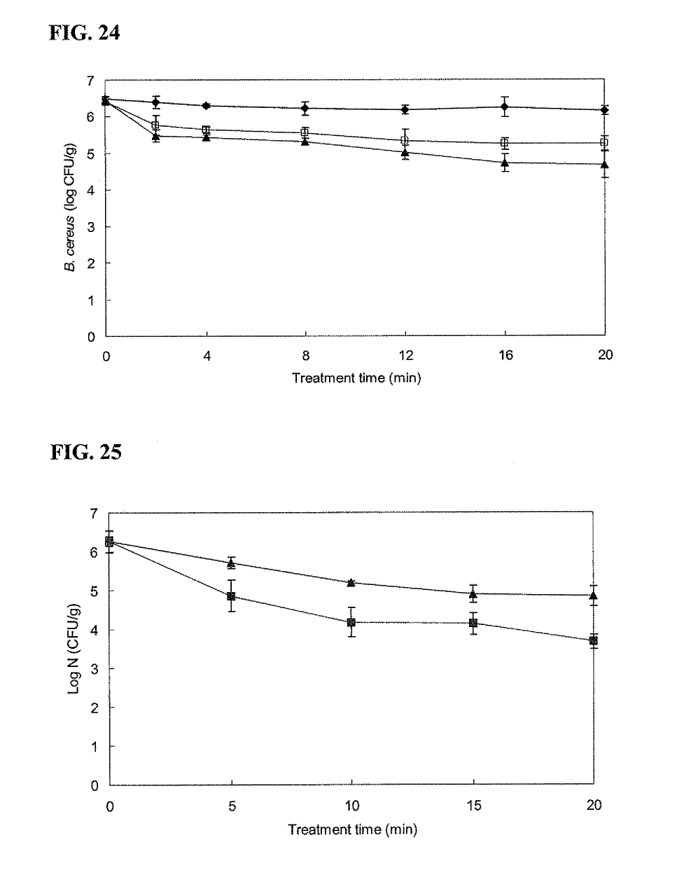

As shown in FIG. 24, when it was treated with only ultraviolet in case of Bacillus cereus inoculated into carrot, the bacteria were reduced by 0.5 log CFU/g after 4 minutes and reduced by 1.2 log CFU/g after 20 minutes. However, when it was carried out together with the photo-catalyst treatment, it was reduced by 1.0 log CFU/g after 4 minutes and after 20 minutes the reducing effect of 2.3 log CFU/g was represented. That is, it could be noted that in only 20 minutes, about 99.9% of bacteria was reduced.

As shown in FIG. 25, when it was treated with only ultraviolet in case of Escherichia coli inoculated into iceberg lettuce, the bacteria were reduced by 0.6 log CFU/g after 5 minutes and reduced by 1.4 log CFU/g after 20 minutes. However, when it was carried out together with the photo-catalyst treatment, it was reduced by 1.4 log CFU/g after 5 minutes and after 20 minutes, the reducing effect of 2.6 log CFU/g was represented. That is, it could be noted that in only 20 minutes, about 99.9% of bacteria was reduced.

As shown in FIG. 26, when it was treated with only ultraviolet in case of Listeria monocytogenes inoculated into iceberg lettuce, the bacteria were reduced by 0.5 log CFU/g after 5 minutes and reduced by 1.0 log CFU/g after 20 minutes. However, when it was carried out together with the photo-catalyst treatment, it was reduced by 1.0 log CFU/g after 5 minutes and after 20 minutes the reducing effect of 2.5 log CFU/g was represented. That is, it could be noted that in only 20 minutes, about 99.9% of bacteria was reduced.

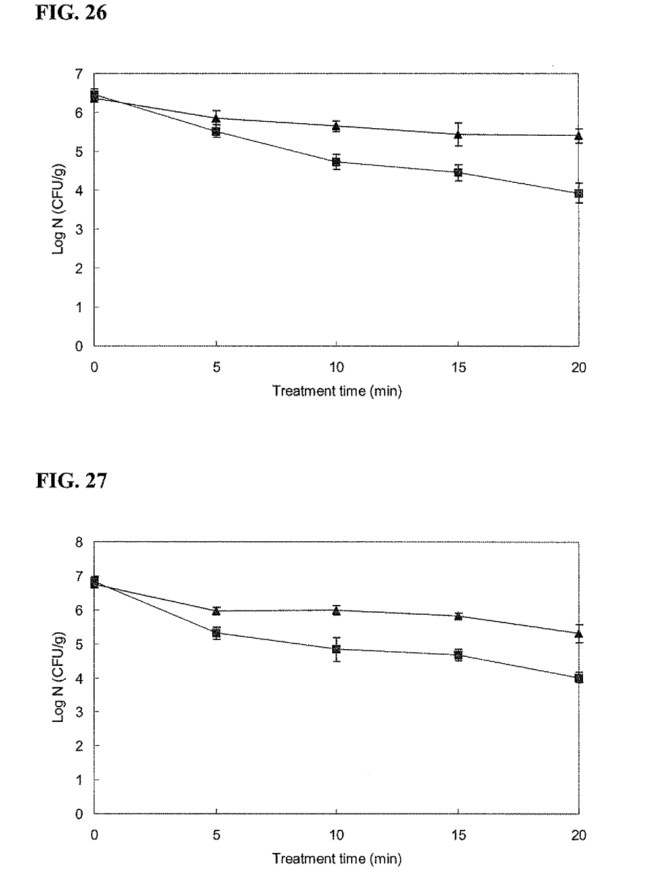

As shown in FIG. 27, when it was treated with only ultraviolet in case of Salmonella typhimurium inoculated into iceberg lettuce, the bacteria were reduced by 0.7 log CFU/g after 5 minutes and reduced by 1.4 log CFU/g after 20 minutes. However, when it was carried out together with the photo-catalyst treatment, it was reduced by 1.5 log CFU/g after 5 minutes and after 20 minutes, the reducing effect of 2.8 log CFU/g was represented. That is, it could be noted that in only 20 minutes, about 99.9% of bacteria was reduced.

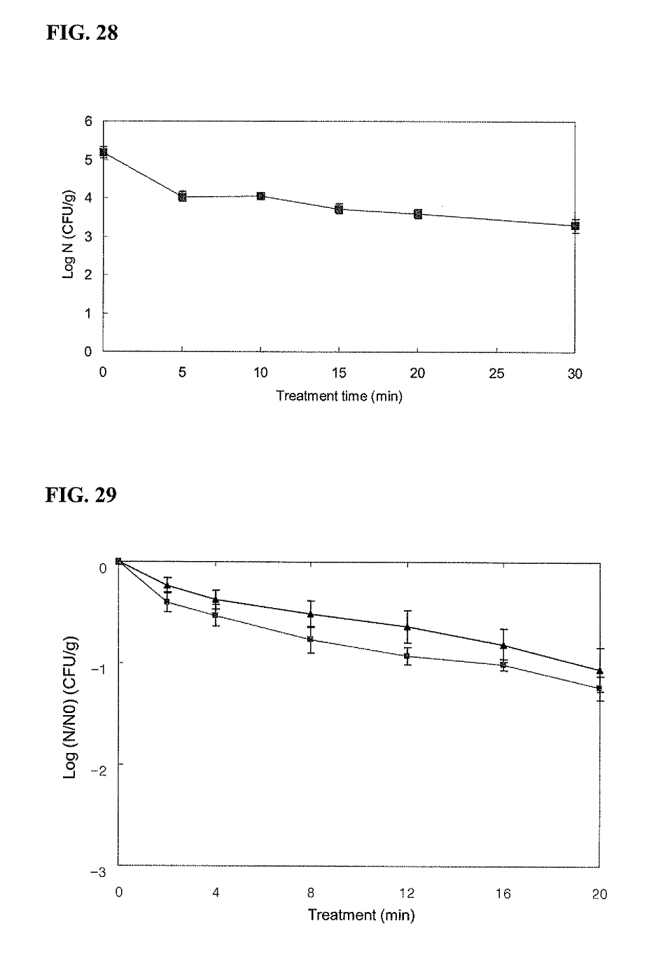

As shown in FIG. 28, when it was treated with the photo-catalyst in case of Bacillus subtilis spores inoculated into ginseng, the bacteria were reduced by 1.2 log CFU/g in only 5 minutes and after 30 minutes, the reducing effect of 1.9 log CFU/g was represented. That is, it could be noted that in only 30 minutes, about 99% of bacteria was reduced.

It could be identified through the present experimental example that when the photo-catalyst disinfecting method was applied to a pre-treatment step, for example, a washing step, and the like, in which microorganisms were reduced for maintaining food freshness and extending food shelf life after harvesting raw materials of fresh foods and health foods, even in case of causing microorganism contamination to them, that the heat treatment was impossible, the effect was very good.

### EXAMPLE 7

### Bactericidal Effect of Photo-Catalyst Disinfecting Apparatus According to pH Against Bacteria Present in Fresh Foods

The effect according to pH against total aerobic bacteria present in raw materials of fresh foods, broccoli, in the photo-catalyst reaction using titanium dioxide. To measure the bactericidal effect against total aerobic bacteria present in broccoli, 2 kg of broccoli cut with certain size was dipped into the photo-catalyst disinfecting apparatuses set up to pH 5.5, 7.5, and 9.5, respectively, and the change of total aerobic bacteria count was measured over time.

Samples were weighed, placed in a stomacher plastic bag (Nasco Whirl-pak filter bag, USA), diluted ten (10) times, and homogenized for 120 seconds using a stomacher (AES MIX-2, France). Then, a liquid phase portion was taken and diluted step by step to inoculate the specimen into the nutrient agar (Difco), and culture it at 35° C. for 24 hours. Then, bacteria number was counted to measure the change of bacteria count according to the reaction time of the photo-catalyst apparatus. The experimental results are represented in FIGS. 29 to 31.

As shown in FIG. 29, when total aerobic bacteria present in broccoli was treated at an acidic condition of pH 5.5 with only ultraviolet, the bacteria were reduced by 0.4 log CFU/g after 4 minutes and reduced by 1.0 log CFU/g after 20 minutes. However, when it was carried out together with the photo-catalyst treatment, it was reduced by 0.6 log CFU/g after 4 minutes and reduced by 1.2 log CFU/g after 20 minutes. That is, it could be noted that in only 20 minutes, about 94% of bacteria was reduced.

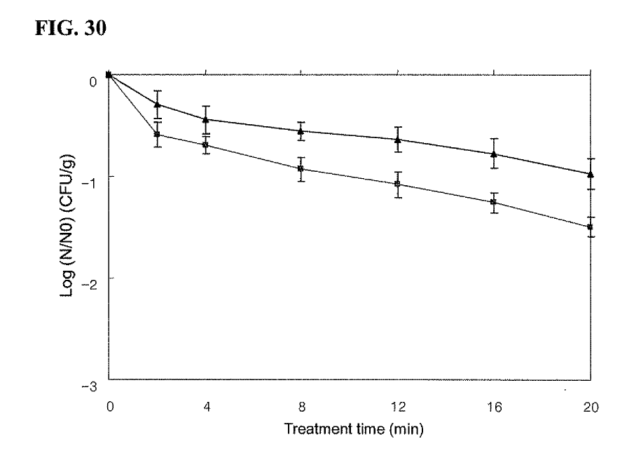

As shown in FIG. 30, when total aerobic bacteria present in broccoli was treated at a neutral condition of pH 7.5 with only ultraviolet, the bacteria were reduced by 0.5 log CFU/g after 4 minutes and reduced by 0.9 log CFU/g after 20 minutes. However, when it was carried out together with the photo-catalyst treatment, it was reduced by 0.7 log CFU/g after 4 minutes and reduced by 1.5 log CFU/g after 20 minutes. That is, it could be noted that in only 20 minutes, about 97% of bacteria was reduced.

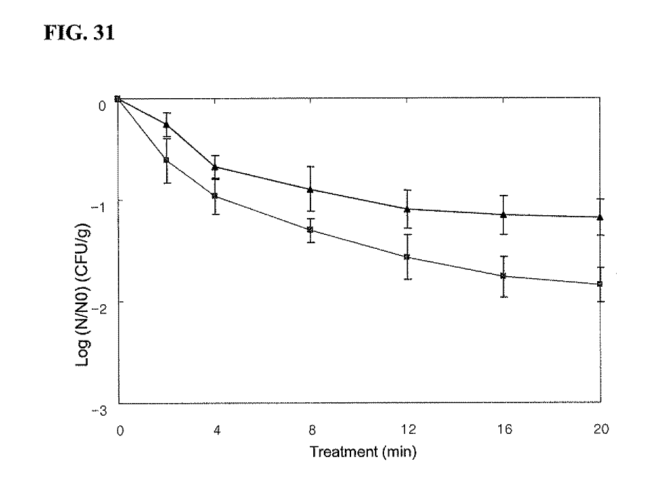

As shown in FIG. 31, when total aerobic bacteria present in broccoli was treated at a basic condition of pH 9.5 with only ultraviolet, the bacteria were reduced by 0.7 log CFU/g after 4 minutes and reduced by 1.0 log CFU/g after 20 minutes. However, when it was carried out together with the photo-catalyst treatment, it was reduced by 1.0 log CFU/g after 4 minutes and reduced by 1.8 log CFU/g after 20 minutes. That is, it could be noted that in only 20 minutes, about 99% of bacteria was reduced.

It could be identified through the present experimental example that when the photo-catalyst disinfecting method was applied to raw materials of fresh foods and health foods, and the like, that the heat treatment was impossible, the effect was better in a basic condition over an acidic condition.

### EXAMPLE 8

### Bactericidal Effect of Photo-Catalyst Disinfecting Apparatus According to pH Against Bacteria Inoculated into Fresh Foods

Harmful microorganisms were inoculated into fresh food raw materials, broccoli, and the bactericidal effect of the photo-catalyst disinfecting apparatus according to pH was identified. To measure the bactericidal effect against E. coli, and Salmonella typhimurium inoculated into broccoli, 2 kg of broccoli cut with certain size was sterilized through ultraviolet irradiation for 30 minutes and was immersed into each bacteria solution. Then, these solutions were dipped into the photo-catalyst disinfecting apparatus at pH 5.5, 7.5 and 9.5 and then the change of bacteria count was measured over time.

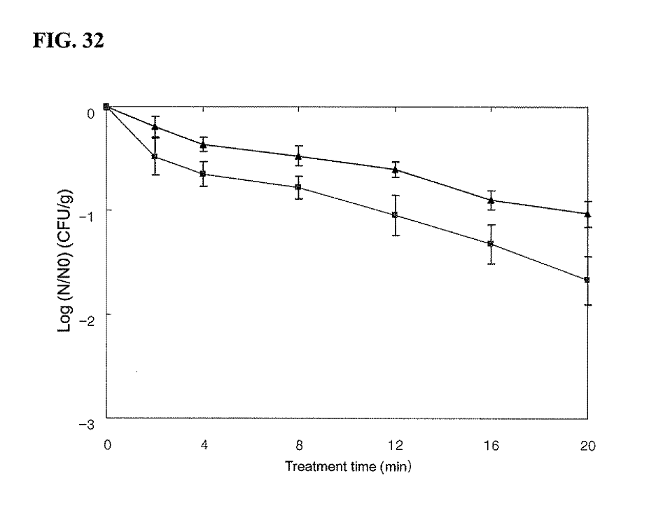

Samples were weighed, placed in a stomacher plastic bag (Nasco Whirl-pak filter bag, USA), diluted ten (10) times, and homogenized for 120 seconds using a stomacher (AES MIX-2, France). Then, a liquid phase portion was taken and diluted step by step to inoculate the specimen into the same medium as Example 4 above by standard plate count method, and culture it at 35° C. for 24 hours. Then, bacteria number was counted to measure the change of bacteria count according to the reaction time of the photo-catalyst apparatus. The experimental results are represented in FIGS. 32 to 37.

As shown in FIG. 32, when it was treated at an acidic condition of pH 5.5 with only ultraviolet in case of E. coli inoculated into broccoli, the bacteria were reduced by 0.4 log CFU/g after 4 minutes and reduced by 1.0 log CFU/g after 20 minutes. However, when it was carried out together with the photo-catalyst treatment, it was reduced by 0.7 log CFU/g after 4 minutes and reduced by 1.7 log CFU/g after 20 minutes. That is, it could be noted that in only 20 minutes, about 98% of bacteria was reduced.

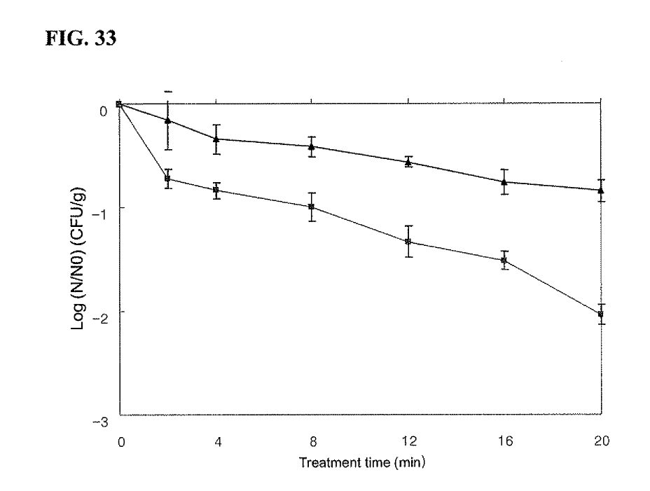

As shown in FIG. 33, when it was treated at a neutral condition of pH 7.5 with only ultraviolet in case of E. coli inoculated into broccoli, the bacteria were reduced by 0.4 log CFU/g after 4 minutes and reduced by 0.8 log CFU/g after 20 minutes. However, when it was carried out together with the photo-catalyst treatment, it was reduced by 0.8 log CFU/g after 4 minutes and reduced by 2.0 log CFU/g after 20 minutes. That is, it could be noted that in only 20 minutes, about 99.9% of bacteria was reduced.

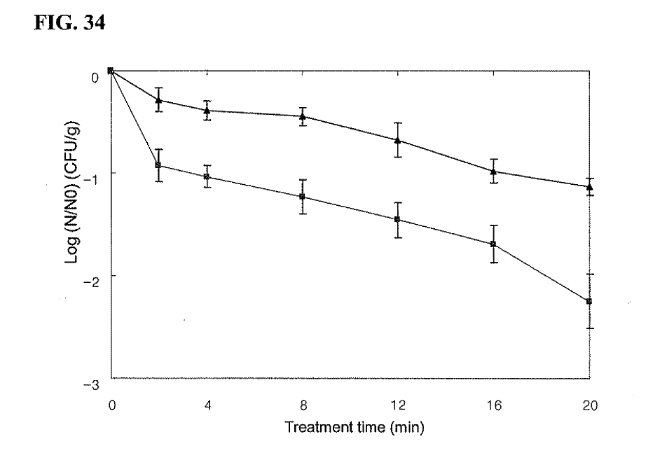

As shown in FIG. 34, when it was treated at a basic condition of pH 9.5 with only ultraviolet in case of E. coli inoculated into broccoli, the bacteria were reduced by 0.4 log CFU/g after 4 minutes and reduced by 1.1 log CFU/g after 20 minutes. However, when it was carried out together with the photo-catalyst treatment, it was reduced by 1.0 log CFU/g after 4 minutes and reduced by 2.3 log CFU/g after 20 minutes. That is, it could be noted that in only 20 minutes, about 99.9% of bacteria was reduced.

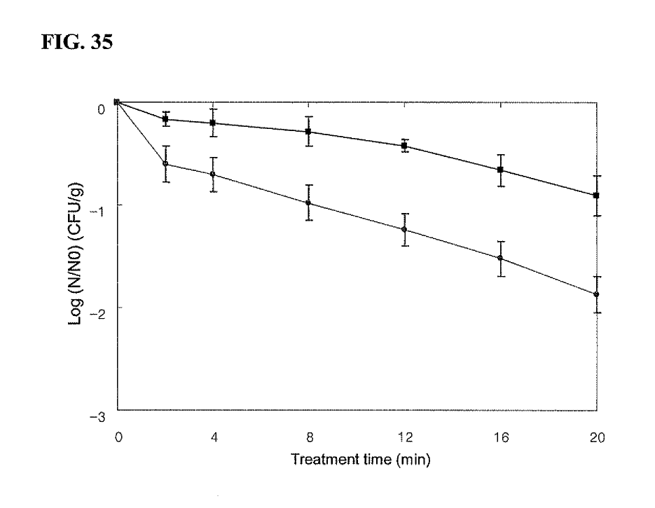

As shown in FIG. 35, when it was treated at an acidic condition of pH 5.5 with only ultraviolet in case of Salmonella typhimurium inoculated into broccoli, the bacteria were reduced by 0.3 log CFU/g after 4 minutes and reduced by 0.9 log CFU/g after 20 minutes. However, when it was carried out together with the photo-catalyst treatment, it was reduced by 0.7 log CFU/g after 4 minutes and reduced by 1.8 log CFU/g after 20 minutes. That is, it could be noted that in only 20 minutes, about 98% of bacteria was reduced.

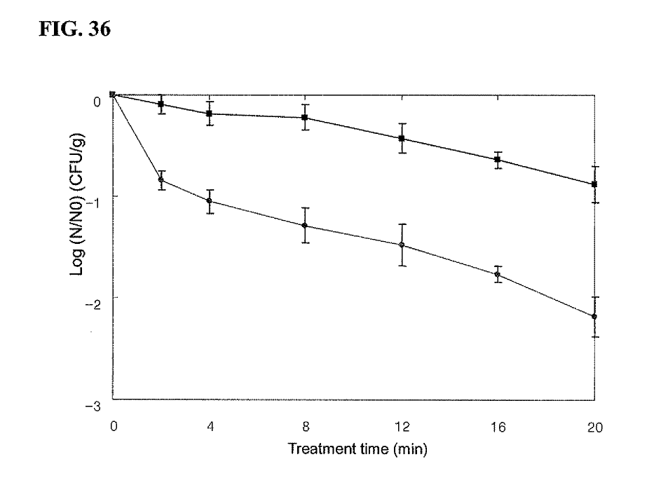

As shown in FIG. 36, when it was treated at a neutral condition of pH 7.5 with only ultraviolet in case of Salmonella typhimurium inoculated into broccoli, the bacteria were reduced by 0.3 log CFU/g after 4 minutes and reduced by 0.8 log CFU/g after 20 minutes. However, when it was carried out together with the photo-catalyst treatment, it was reduced by 1.0 log CFU/g after 4 minutes and reduced by 2.3 log CFU/g after 20 minutes. That is, it could be noted that in only 20 minutes, about 99.9% of bacteria was reduced.

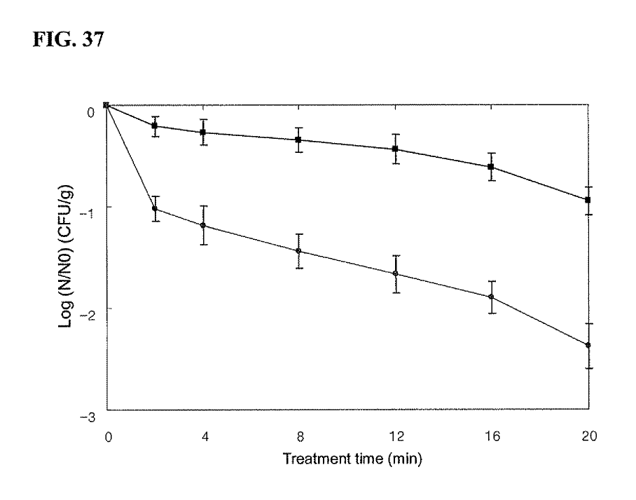

As shown in FIG. 37, when it was treated at a basic condition of pH 9.5 with only ultraviolet in case of Salmonella typhimurium inoculated into broccoli, the bacteria were reduced by 0.3 log CFU/g after 4 minutes and reduced by 1.0 log CFU/g after 20 minutes. However, when it was carried out together with the photo-catalyst treatment, it was reduced by 1.2 log CFU/g after 4 minutes and reduced by 2.4 log CFU/g after 20 minutes. That is, it could be noted that in only 20 minutes, about 99.9% of bacteria was reduced.

It could be identified through the present experimental example that when the photo-catalyst disinfecting method was applied to raw materials of fresh foods, and the like, that the heat treatment was impossible, the effect was better in a basic condition over an acidic condition.

### EXAMPLE 9

### Microbial Quality of Fresh Produce Treated with Photo-Catalyst Disinfecting Apparatus During Storage

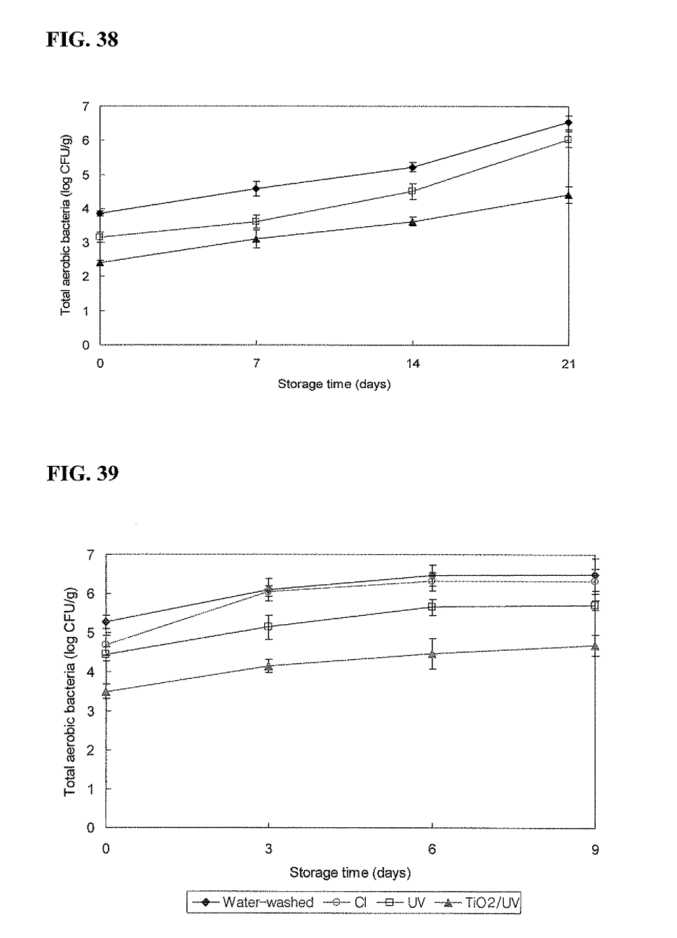

Effects of the photo-catalytic disinfection on microbial quality of fresh carrot and iceberg lettuce were measured for up to 21 days and 9 days, respectively, according to Example 5 above. The results are represented in FIGS. 38 and 39.

As shown in FIG. 38, the population of total aerobic bacteria on carrots increased over the storage period. The initial counts of total aerobic bacteria in fresh carrots were reduced to 2.4, 3.1, and 3.9 log CFU/g, respectively, after titanium dioxide-UV, UV only, and tap water treatment. Total aerobic bacterial population in titanium dioxide-UV treated fresh carrots increased to 4.4 log CFU/g after 21 day storage at 4° C., while the number of total aerobic bacteria in UV-treated and tap water-treated fresh carrots increased to 6.0 and 6.5 log CFU/g after the storage.

As shown in FIG. 39, the population of total aerobic bacteria on iceberg lettuce increased over the storage period. The counts of total aerobic bacteria in fresh iceberg lettuce were 3.4, 4.5, and 4.7 log CFU/g, respectively, after titanium dioxide-UV, UV only, and chlorine treatment. Total aerobic bacterial population in titanium dioxide-UV treated fresh iceberg lettuce increased to 4.6 log CFU/g after 9 day storage at 4° C., while the number of total aerobic bacteria in UV-treated and chlorine-treated fresh iceberg lettuce increased to 5.8 and 6.3 log CFU/g after the storage. From both results the photo-catalyst treatment clearly showed a lower growth rate than that resulting from other treatments.

It could be identified through the present example that the photo-catalyst treatment can be used to extend shelf-life of fresh foods and the like and improve microbial quality during storage.

The disinfecting apparatus according to the present invention, generating the photo-catalytic reaction by forming the photo-catalyst layer of titanium dioxide on the outer surface of quartz tube and setting the ultraviolet lamp inside the quartz tube may be utilized as a disinfecting apparatus for reducing harmful microorganisms which are problematic in foods. Since radicals made by the photo-catalytic reaction have high reactivity and strong bactericidal capacity, they serve to sterilize microorganisms in a short time.

The disinfecting apparatus using such ultraviolet titanium dioxide photo-catalyst and the method thereof have excellent bactericidal capacity, and are economical and safe. Particularly, they may be effectively employed in a pre-treatment step, for example, a washing step and the like, in which microorganisms are reduced for maintaining food freshness and extending food shelf life after harvesting fresh foods and the like that the heat treatment is impossible.

Although the specific embodiment has been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and appended claims. In addition to variations and modifications in the component parts and/or arrangements, alternative uses will also be apparent to those skilled in the art.

1. An apparatus for disinfecting an object, comprising: a bath in which water is flowed in and drained out; and at least one ultraviolet ray (UV) lamp unit assembly mounted in the bath, wherein each UV lamp unit assembly comprises a plurality of UV lamp units and each of the UV lamp units comprises a quartz tube, a UV lamp mounted in the quartz tube and a photo-catalyst layer of titanium dioxide formed on an outer surface of the quartz tube to disinfect objects in the bath by the photo-catalytic reaction of titanium dioxide and UV.

2. The apparatus according to claim 1, wherein the bath comprises a case in which water is accommodated and a cover mounted openably/closably to the case, and the UV lamp unit assembly is provided at a lower portion of the bath.

3. The apparatus according to claim 2, further comprising an additional UV lamp unit assembly provided on an inner surface of the cover.

4. The apparatus according to claim 1, further comprising an air-blowing means including an air flow line provided below the UV lamp unit assembly, an air supplying unit installed outside the bath and a connecting line connecting the air flow line and the air supplying unit so as to inject air into water in the bath through openings formed on the air flow line.

5. The apparatus according to claim 1, further comprising: a base including a plurality of guide channels fixed thereon and a driving means mounted on the base for reciprocating the bath linearly; a plurality of roller fixed rotatably on a lower surface of the bath and received in the guide channels of the base; and a fixture fixed to lower surface of the bath and connected to the driving means of the base.

6. The apparatus according to claim 1, further comprising a conveyer device comprising a driving roller, driven rollers and a conveyer belt wound around the rollers, the conveyer device being divided into an inlet portion formed at a first outside of the bath, a conveying portion formed in water in the bath and a discharging portion formed at a second outside of the bath, wherein the bath has openings formed on the first and second side walls thereof for enabling the conveyer and object putted on the conveyer to be passed through the openings, and the UV lamp unit assembly is disposed below the conveyer belt in the bath.

7. The apparatus according to claim 6, wherein the UV lamp unit assembly is disposed between an upper portion and a lower portion of the conveyer belt.

8. The apparatus according to claim 7, wherein the conveyer belt includes a pair of supporting bodies spaced apart from each other and a mesh sheet secured to the supporting bodies, and the supporting bodies are supported on idle roller provided at walls of the bath.

9. The apparatus according to claim 8, wherein the mesh sheet comprises a plurality of pins secured to an outer surface thereof.

10. The apparatus according to claim 1, further comprising a first conveyer device provided in the bath and a second conveyer device placed below the first conveyer device, each of the first and second conveyer devices comprising a driving roller, driven rollers and a conveyer belt wound around the rollers, and each conveyer device being divided into an inlet portion formed at a first outside of the bath, a conveying portion formed in water in the bath and a discharging portion formed at a second outside of the bath, wherein the bath has openings formed on the first and second side walls thereof for enabling the conveyer and object putted on the conveyer to be passed through the openings, and each UV lamp unit assembly is disposed below the corresponding conveyer belt in the bath.

11. The apparatus according to claim 10, wherein each of the UV lamp unit assemblies is disposed between an upper portion and a lower portion of the corresponding conveyer belt.

12. The apparatus according to claim 10, wherein the conveyer belt of each conveyer device includes a pair of supporting bodies spaced apart from each other and a mesh sheet secured to the supporting bodies, and the supporting bodies are supported on idle roller provided at walls of the bath.

13. The apparatus according to claim 12, wherein the mesh sheet comprises a plurality of pins secured to an outer surface thereof.

14. A method of disinfecting an object, which is performed by utilizing the device according to claim 1.
